(19) United States
(12) Patent Application Publication
(10) Pub. No.: US 2022/0135953 A1
(43) Pub. Date: May 5, 2022

(54) BIOFUNCTIONALIZED HYDROGEL FOR CELL CULTURE

(71) Applicant: Vanderbilt University, Nashville, TN (US)

(72) Inventors: Ethan S. Lippmann, Nashville, TN (US); Kylie Balotin, Nashville, TN (US); Brian O'Grady, Nashville, TN (US); Leon M. Bellan, Nashville, TN (US)

(21) Appl. No.: 17/429,540

(22) PCT Filed: Feb. 21, 2020
(86) PCT No.: PCT/US2020/019369

§ 371 Date: Aug. 9, 2021

Related U.S. Application Data
(60) Provisional application No. 62/809,184, filed on Feb. 22, 2019.
(60) Provisional application No. 62/828,806, filed on Apr. 3, 2019.
(60) Provisional application No. 62/857,575, filed on Jun. 5, 2019.

(51) Int. Cl. C12N 5/071; C12N 5/0793; C12N 5/079; C08J 3/075; C08J 3/24; C08H 1/06; C08L 89/06

(52) U.S. Cl. CPC C12N 5/0697; C08L 2312/00; C12N 5/0622; C12N 5/0691; C08J 3/075; C08J 3/24; C08H 1/06; C08L 89/06; C12N 2537/10; C12N 2513/00; C12N 2533/54; C12N 2502/081; C12N 2502/086; C12N 2506/45; C08J 2389/06; C12N 5/0619

(57) ABSTRACT

Provided are biomaterials useful for cell culture, method of preparation thereof, and use thereof. The present biomaterial comprises a crosslinked hydrogel and a peptide chemically attached to the hydrogel, wherein the peptide comprises a histidine-alanine-valine (HAV) sequence. In particular, the present biomaterial may be useful for culturing neurons, brain endothelial cells, and/or glial cells, supporting the formation of synaptically connected neural networks, and growing stem cell-derived organoids that more closely resemble human organs.

### CROSS-REFERENCE TO RELATED APPLICATIONS

This application claims the benefit of and priority to U.S. Provisional Application No. 62/809,184, filed on Feb. 22, 2019, U.S. Provisional Application No. 62/828,806, filed on Apr. 3, 2019, and U.S. Provisional Application No. 62/857,575, filed on Jun. 5, 2019, the entire contents of which are hereby incorporated by reference.

### STATEMENT OF GOVERNMENT INTEREST

This invention was made with government support under grants 1462866 and 1506717 awarded by the National Science Foundation. The government has certain rights in the invention.

### SEQUENCE LISTING

The instant application includes a Sequence Listing which has been submitted electronically in ASCII format and is hereby incorporated by reference in its entirety. Said ASCII copy, created on Feb. 21, 2020, is named 093386-9267-WO01_As_Filed_Sequence_Listing and is 879 bytes in size.

### INTRODUCTION

Neurodegenerative diseases (e.g. Alzheimer's disease, Parkinson's disease, Huntington's disease, Amyotrophic Lateral Sclerosis and Multiple Sclerosis) all have different region-specific presentation and modes of cell-cell communication. These differences make it difficult to understand the mechanisms underlying the onset and propagation of neurodegenerative disorders and thereby hamper the development of effective treatments. Many candidate therapeutics that are efficacious in various mouse models of neurodegenerative diseases are not efficacious in humans. As such, there is a critical need to develop human in vitro models for studying neurodegeneration and to increase the translatability of therapeutic development research.

Recent advancements in three-dimensional (3D) neural tissue models, particularly those constructed from human pluripotent stem cell (hPSC)-derived progenies (including human embryonic stem cells and induced pluripotent stem cells (iPSCs)), have the ability to mimic the structure and function of human brain regions. Such models typically consist of neurons and varying mixtures of supporting cells (e.g. glia and vascular components) embedded in a hydrogel formed from extracellular matrix (ECM) components. The majority of early neural tissue models have utilized Matrigel, an ECM composite derived from Engelbreth-Holm-Swarm mouse sarcoma tumors that consists of proteins (e.g. type IV collagen, laminin) and growth factors. For example, 3D Matrigel scaffolds has been used to support differentiation of mouse embryonic stem cells to neural cells. Matrigel is also the sole ECM currently utilized for hPSC-derived brain organoids, where the ECM scaffold supports the self-organization of the neuroepithelium to induce neuroepithelial buds and facilitates growth by providing a physical structure for cells to attach to and grow. Other natural and synthetic materials have been developed for extended culture of hPSC-derived neural progenitor cells (NPCs) and neurons, including silk, collagen, hyaluronic acid (HA), elastin-like peptides, and polyethylene glycol (PEG). As these materials all allow for diffusion of essential nutrients and morphogens throughout the tissue constructs, they can be used to maintain NPCs and neuronal cultures for extended studies of differentiation and maturation, including axon formation, growth, and pruning. Additionally, these platforms have demonstrated utility for assessing disease phenotypes when the hPSCs are sourced from patients that harbor genetic risk factors for each disorder.

Despite progress towards fabricating complex neural tissue structures from hPSCs, existing ECMs have many shortcomings for practical neural cell culture. One limitation of existing ECM platforms is the lack of appropriate bioinstructive cues to promote cell-cell or cell-ECM interactions that facilitate neuronal maturation. Of the aforementioned materials, only Matrigel (e.g. laminin) and HA have physiological relevance to brain ECM; HA in particular has been shown to support hPSC-derived NPC maturation into neurons that exhibit enhanced neurite projection with synaptic vesicles and electrophysiological activity. However, Matrigel and HA are difficult to handle due to their viscosity, and both materials have a very low elastic modulus, meaning they collapse under their own weight and cannot be molded into more complex structures. These factors limit the fabrication of topographic features, such as vasculature or perfusion channels. Synthetic hydrogels may overcome these issues by incorporating custom functional groups that enable tuning of mechanical and rheological properties, but they can be prohibitively difficult to fabricate and require extensive chemical modification to recapitulate tissue-specific biochemical cell-ECM interactions. Moreover, the majority of natural and synthetic ECMs are relatively expensive, which can further limit their widespread use.

Thus, there remains a need for an ECM material that facilitates neural tissue survival and maturation within 3D tissue constructs through biophysical cues, exhibit ideal mechanical properties to promote neural and vascular outgrowth while also supporting micropatterned features, and be relatively easy to synthesize, low cost and therefore widely accessible.

### SUMMARY

In one aspect, the present disclosure provides biomaterial comprising a crosslinked hydrogel and a peptide chemically attached to the hydrogel, wherein the peptide comprises a histidine-alanine-valine (HAV) sequence.

In another aspect, the present disclose provides a method of preparing a biomaterial, comprising:

chemically attaching a peptide comprising a histidine-alanine-valine (HAV) sequence to a hydrogel;

chemically attaching a crosslinker to the hydrogel; and

crosslinking the hydrogel having the attached peptide and the attached crosslinker.

In yet another aspect, the present disclosure provides a method of culturing a plurality of cells, comprising contacting the plurality of cells with the biomaterial as described herein. In some embodiments, the present biomaterial is used for culturing neurons, brain endothelial cells, glial cells, or combinations thereof.

### BRIEF DESCRIPTION OF THE DRAWINGS

FIG. 1 shows a schematic illustration of GelMA synthesis and N-cadherin peptide conjugation. The conventional method for synthesizing GelMA uses methacrylic anhydride to introduce a methacryloyl substitution group on the reactive primary amine group of amino acid residues. GelMA was then dissolved in TEAO buffer with the N-cadherin peptide for Michael-type addition to the reactive primary amine group of the amino acid.

FIG. 2 shows an assessment of biomaterial functionalization and physical properties of polymerized hydrogels. (B) NMR spectra of gelatin, GelMA, GelMA-Cad, and GelMA-Scram. Successful conjugation of methacrylic anhydride to the backbone of gelatin was assessed by peaks at 5.5 and 5.7 ppm, and N-cadherin/Scram peptide addition was assessed by the valine peak at 3.5 ppm. (C) FTIR spectra was used to confirm conjugation of the peptide to the backbone of GelMA due to decrease in the following relevant bands: 1000 cm-1 (PO4 stretching) and 1250 cm-1, 1540 cm-1, and 1640 cm-1 (NH bending). (D) AFM measurements of Young's modulus values for GelMA, GelMA-Cad, GelMA-Scram. Data are presented as mean±S.D. from 3 independently fabricated hydrogels, where three locations were sampled on each hydrogel as described in the methods.

FIG. 3 shows an assessment of patterned architectures in hydrogels fabricated from GelMA-Cad or Matrigel. PDMS molds were filled with GelMA-Cad or Matrigel and crosslinked around a piece of silicone tubing, which was then manually removed. (A) GelMA-Cad hydrogel shows an intact channel that can be perfused. (B) The channel in the Matrigel hydrogel collapses after the tubing is removed.

FIG. 4 shows SEM images of hydrogels fabricated from gelatin, GelMA, GelMA-Cad, and GelMA-Scram.

FIG. 5 shows live/dead staining of iPSC-derived neurons embedded in various hydrogels. For panels A-H, cells were labeled with calcein to visualize live cells and propidium iodide (PI) to visualize dead cells. For panels A-B, both calcein and PI staining are shown to highlight dead cells. For panels C-H, only calcein is shown to highlight neuron morphology in GelMA-Cad, and insets are provided for higher magnification. Full quantification of viability is shown in panel L. (A) Neurons in GelMA 48 hours after embedding. (B) Neurons in GelMA-Scram 48 hours after embedding. (C-D) Neurons in Matrigel or GelMACad 48 hours after embedding. (E-F) Neurons in Matrigel or GelMA-Cad 72 hours after embedding. (G-H) Neuron in Matrigel or GelMA-Cad 7 days after embedding. (I-K) Neurons in GelMA-Cad were immunolabeled 7 days after embedding for βIII tubulin to confirm identity. (L) Cell viability is presented for various time points as mean±S.D. from 3 biological replicates, with 5 images assessed per replicate.

FIG. 6 shows an assessment of cell viability in iPS C-derived neurons embedded in GelMA or Matrigel with soluble peptides. For panels A-D, cells were labeled with calcein to visualize live cells and propidium iodide (PI) to visualize dead cells. All images were taken 4 days after embedding. (A) Neurons embedded in GelMA with soluble N-cadherin peptide. (B) Neurons embedded in GelMA with soluble scrambled peptide. (C) Neurons embedded in Matrigel with soluble N-cadherin peptide. (D) Neurons embedded in Matrigel with soluble scrambled peptide. (E) Quantification of cell viability. Data represent mean±S.D. from 3 biological replicates, with 4 images assessed per replicate.

FIG. 7 shows quantification of neurites in iPSC-derived neurons embedded in Matrigel and GelMA-Cad. Panels A-C demonstrate the quantification of neurites in GelMA-Cad on day 5, and panels D-E demonstrate the quantification of neurites in GelMA-Cad on day 10. Neurons are stained with calcein (green) and imaged with a confocal microscope (A, D). Using custom Matlab code, a mask is applied (B, E) and cell soma and neurites are identified (C,F), where red corresponds to the soma and green corresponds to neurite extensions, which can then be measured and averaged across an image. (G-H) Example of high resolution images of neurites in GelMA-Cad and Matrigel, where differences in neurite length and thickness can be observed. (I-J) Full quantification of neurite length and width. Data are presented as mean±S.D. from 7 biological replicates, with 4 images quantified per replicate. Statistical significance was calculated using the student's unpaired t-test (*, p<0.05).

FIG. 8 shows iPSC-derived astrocytes respond well to GelMA-Cad hydrogel: markers are GFAP (red), actin (green), and DAPI nuclear stain (blue). Astrocytes in GelMA-Cad (A) extend their processes and have minimal GFAP expression, indicating quiescence and maturity. Image (B) is astrocytes in GelMA-Cad treated with TNF-alpha to activate inflammation (positive control).

FIG. 9 shows an assessment of synaptic connectivity of iPSC-derived neurons in Matrigel or GelMA-Cad by immunostaining and electrophysiology. (A) Immunostaining of synaptophysin and PSD-95 in neurons that were embedded in each hydrogel for 21 days. An inset is provided to highlight pronounced differences. 10 images from 3 biological replicates were used for absolute quantification of expression and percent co-localization. (B) Electrical activity in neurons embedded in GelMA-Cad (red) and Matrigel (black) for 21 days. Voltage traces are representative of 5 biological measurements.

FIG. 10 shows an assessment of synaptic connectivity of iPSC-derived neurons in Matrigel or GelMA-Cad by viral tracing. The schematic depicts the experimental approach, where wild-type neurons were uniformly mixed in a hydrogel and a small population of AAV-transduced neurons were injected into the center. EGFP was then imaged at day 7 and day 21. Calcein was added at day 21 to verify cell viability as highlighted by the insets. The images from these experiments are representative of 6 biological replicates that confirmed the transmission of EGFP in GelMA-Cad but not Matrigel.

FIG. 11 shows formation of junctions between endothelial cells and that Gel-MA-Cad supports maintenance of their cellular phenotype. Gel-MA-Cad prevents brain endothelial cells (BMECs) generated from iPSCs from de-differentiating and losing their vascular phenotype, as denoted by maintenance of VE-cadherin expression (green) in the cell junctions. iPSCs were differentiated to BMECs according to established methods (A) and then purified for extended culture on plastic dishes with or without GelMA-Cad (B-D).

FIG. 12 shows significant vascular growth in primary brain tissue. Brightfield images show that new vessels only sprout in GelMA-Cad, not Matrigel (A-C). Sprouted vessels include (D) arteries (larger vessels with multiple claudin-5-positive endothelial cells lined by smooth muscle [SMA=smooth muscle actin]) and (E) capillaries consisting of single lumen (occluding-positive endothelial cells lined with a single layer of NG2-positive pericytes).

FIG. 13 shows (A) Brain organoids differentiated from iPSCs embedded in GelMA-Cad or Matrigel. Brain organoids embedded in GelMA-Cad show uniform spherical compaction whereas Matrigel yields organoids with many disorganized neuroepithelial buds. (B) Brain organoids embedded in GelMA-Cad exhibit laminar patterning of deep cortical layers as marked by distinct regions of TBR1 and CTIP2. (C) Brain organoids embedded in GelMA-Cad exhibit robust neuronal outgrowth.

FIG. 14 shows a schematic illustration of a process for preparing a hydrogel (gelatin) with attached peptide and a crosslinker (HPA).

FIG. 15 shows representative NMR spectra of gelatin, Gel-Cad, and Gel-Cad-HPA.

FIG. 16 shows sprouted vessels from primary human brain tissue embedded in redox-crosslinking hydrogel. (A) 10× magnification of brain tissue vessels in (B). (B) Brain tissue vessels marked by Calcein-AM 24 hours after embedding in the hydrogel. (C) 10× magnification of brain tissue vessels in (D). (D) Brain tissue vessels marked by Calcein-AM 48 hours after embedding in the hydrogel. (E) 10× magnification of brain tissue vessels in (F). (F) Brain tissue vessels marked by Calcein-AM 4 days after embedding in the hydrogel.

### DETAILED DESCRIPTION

The present disclosure relates to biomaterials that may be use for culturing cells, in particular neurons and brain cells. The biomaterials may be prepared by chemically attaching a peptide to a hydrogel and crosslinking the hydrogel by using a crosslinker. In particular embodiments, the peptide comprises an N-cadherin extracellular peptide epitope and the biomaterial may maintain a patterned architecture. The biomaterials may promote survival and maturation of neurons, such iPSC-derived glutamatergic neurons, into synaptically connected networks. Given its ability to enhance neuron maturity and connectivity, the biomaterials may be broadly useful for in vitro studies of neural circuitry in health and disease.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl” as used herein, means a straight or branched chain saturated hydrocarbon. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “crosslinker” as used herein refers to a molecule or a function group capable of linking one polymer to another polymer, or one part of a polymer to another part of the polymer, via formation of one or more chemical bonds between the two polymers or the two parts of the polymer.

The term “chemically bonding” or “chemically attaching” as used herein refers to forming a chemical bond between two substances. The chemical bond may be an ionic bond, a covalent bond, dipole-dipole interaction, or hydrogen bond.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. All amino acid residue sequences are represented herein by formulae with left and right orientation in the conventional direction of amino-terminus to carboxy-terminus.

“Substantially identical” means that a first and second amino acid sequences are at least 60%, 65%, 70%, 75%, 80%, 85%, 90%, 95%, 96%, 97%, 98%, or 99% identical over a region of 10, 20, 30, 40, 50, 60, 70, 80, 90, or even 100 amino acids.

A “variant” refers to a peptide or polypeptide that differs in amino acid sequence by the insertion, deletion, or conservative substitution of amino acids, but retain at least one biological activity. Representative examples of “biological activity” include, for example, the ability to promote cell adhesion, to be bound by a specific antibody or polypeptide, or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof. In some embodiments, variants include homologues. Homologues may be polypeptides or genes inherited in two species by a common ancestor.

The term “conservative change” refers to a change made to an amino acid sequence without altering activity. These changes are termed conservative substitutions or mutations; that is, an amino acid belonging to a grouping of amino acids having a particular size or characteristic can be substituted for another amino acid. Substitutes for an amino acid sequence may be selected from other members of the class to which the amino acid belongs. For example, the nonpolar (hydrophobic) amino acids include alanine, leucine, isoleucine, valine, proline, phenylalanine, tryptophan, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such alterations are not expected to substantially affect apparent molecular weight as determined by polyacrylamide gel electrophoresis or isoelectric point. Exemplary conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free NH₂. Moreover, point mutations, deletions, and insertions of the polypeptide sequences or corresponding nucleic acid sequences may in some cases be made without a loss of function of the polypeptide or nucleic acid fragment.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Biomaterial

In one aspect, provided is a biomaterial comprising a crossed hydrogel and a peptide chemically attached to the hydrogel, wherein the peptide comprises a histidine-alanine-valine (HAV) sequence

The present biomaterial may function as an extracellular matrix (ECM) material useful in tissue culture. Suitable hydrogel, peptide, and crosslinker are selected such that the resulting biomaterials as disclosed herein may (1) facilitate cell (such as neurons or brain cells) survival and maturation within 3D tissue constructs through biophysical cues, (2) exhibit ideal mechanical properties to promote neuron outgrowth while also supporting micropatterned features, and/or (3) be relatively easy to synthesize, low cost, and therefore widely accessible.

Hydrogel

The hydrogel may be a polymeric material having a network of hydrophilic polymers. The hydrophilic polymers may be natural or synthetic polymers, and may include known polymers used for tissue engineering, cell culture, biosensors, implants, etc. Suitable hydrogels include hydrogels comprising one or more of hyaluronic acid, polyethylene glycol, polypropylene glycol, polyethylene oxide, polypropylene oxide, polyglutamate, polylysine, polysialic acid, polyvinyl alcohol, polyacrylate, polymethacrylate, polyacrylamide, polymethacrylamide, polyvinyl pyrrolidone, polyoxazoline, polyiminocarbonate, polyamino acid, hydrophilic polyester, polyamide, polyurethane, polyurea, dextran, agarose, xylan, mannan, carrageenan, alginate, gelatin, collagen, albumin, cellulose, methylcellulose, ethylcellulose, hydroxypropylmethylcellulose, hydroxyethyl starch, chitosan, nucleic acids, derivatives thereof, co-polymers thereof, or combinations thereof. Examples of natural hydrogels include those derived from animal tissues, such as gelatin. For example, the hydrogel moiety may be gelatin, or may include a variant or derivative of gelatin. In some embodiments, the hydrogel may include gelatin and one or more other components, such as a hydrophilic polymeric component (e.g. PEG), a hyaluronic acid, or chitosan. In some embodiments, the hydrogel comprises gelatin, such as animal skin gelatin. In particular embodiments, the hydrogel comprises porcine skin gelatin.

Peptide

The peptide may comprise a flanking sequence at the N-terminal end, the C-terminal end, or both the N- and C-terminal ends of the HAV sequence. The peptide may be chemically attached to the hydrogel at the N-terminal end or the C-terminal end. For example, the peptide may be attached to the hydrogel through a residue at the C-terminal end. The amino acid through which the peptide is attached to the hydrogel may be a polar amino acid, such as cysteine (Cys) or glutamic acid (Glu). In some embodiments, the peptide is attached to the hydrogel through a C-terminal Cys or C-terminal Glu. In some embodiments, the peptide is attached to the hydrogel at the C-terminal end, and the N-terminal end of the peptide include a known tag or modification, such as an acetyl group (Ac). In some embodiments, the peptide is attached to the hydrogel via a C-terminal Cys or a C-terminal Glu, and the N-terminal end of the peptide is acetylated.

In some embodiments, the peptide is 5 to 30 amino acids in length. The peptide may include at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, or at least 29 amino acids. The at peptide may include less than 30, less than 29, less than 28, less than 27, less than 26, less than 25, less than 24, less than 23, less than 22, less than 21, less than 20, less than 19, less than 18, less than 17, less than 16, less than 15, less than 14, less than 13, less than 12, less than 11, or less than 10 amino acids. The peptide may be 5 to 25 amino acids in length, 8 to 25 amino acids in length, 8 to 15 amino acids in length, or 8 to 12 amino acids in length. In some embodiments, the peptide is 8 to 12 amino acids in length. In particular embodiments, the peptide is 9 or 10 amino acids in length.

In some embodiments, the peptide is comprises an extracellular epitope of a cadherin protein, or a variant thereof. The term “cadherin” refers to a family of cell surface proteins, which may participate in Ca²⁺-dependent cell adhesion. Some subfamilies of cadherins are considered classical cadherins, which have multiple extracellular domains, a transmembrane domain, and a cytoplasmic domain. Examples of known cadherins include N-cadherin, E-cadherin, and P-cadherin. Sequences of cadherin proteins and variates thereof include those described in Kister et al. (Protein Sci., 2001, 10(9): 1801-1810), Renaud-Young et al. (J. Biol. Chem., 2002, 277(42), 39609-39616), Williams et al. (J Biol Chem., 2002, 277(6), 4361-4367), and Williams et al. (J Biol Chem., 2000, 275(6), 4007-4012), the entire contents of which are incorporated herein by reference.

In some embodiments, the peptide comprises an extracellular epitope of a cadherin protein with one or more conservative changes. In some embodiments, the peptide comprises a sequence that is substantially identical to an extracellular epitope of a cadherin protein. For example, the peptide may comprise a sequence that is at least 85%, at least 90%, at least 91%, at least 92%, at least 93%, at least 94%, at least 95%, at least 96%, at least 97%, at least 98%, or at least 99% identical to an extracellular epitope of a cadherin protein. In some cases a determination of the percent identity of a peptide to a sequence set forth herein (e.g., a Cadherin protein sequence) may be required. In such cases, the percent identity is measured in terms of the number of residues of the peptide, or a portion of the peptide. A peptide of, e.g., 90% identity, may also be a portion of a larger peptide. Embodiments include such peptides that have the indicated identity and/or conservative substitution of a cadherin sequence set forth herein, with said polypeptides exhibiting specific cell adhesion activities.

In some embodiments, the HAV sequence is at the N-terminal end of the peptide. In some embodiments, the peptide further comprises a Asp-Ile-Gly-Gly (DIGG) sequence, a Asp-Ile-Asn-Gly (DING) sequence, a Ser-Ser-Asn-Gly (SSNG) sequence, or a Ser-Glu-Asn-Gly (SENG) sequence. The DIGG, DING, SSNG, or SENG sequence may be to the C-terminal of the HAV sequence. For example, the DIGG, DING, SSNG, or SENG sequence may be attached to the C-terminal end of the HAV sequence.

In some embodiments, the peptide comprises SEQ ID NO: 1 (HAVDIGGGC), SEQ ID NO: 2 (HAVDIGGGCE), or a variant thereof. In some embodiments, the peptide consists of SEQ ID NO: 1, SEQ ID NO: 2, or a variant thereof. In some embodiments, the peptide includes at least one additional amino acid at the C-terminal end, at the N-terminal end, or at both the C-terminal and N-terminal ends, of the amino acid sequence of SEQ ID NO: 1 or SEQ ID NO: 2. In some embodiments, the peptide includes sequence tags or modifications as known in the art to the C-terminal end, the N-terminal end, or both the C-terminal and N-terminal ends of SEQ ID NO: 1 or SEQ ID NO: 2. In some embodiments, the peptide includes an acetyl group (Ac) at the N-terminal end of the amino acid sequence of SEQ ID NO: 1 (Ac-HAVDIGGGC) or SEQ ID NO: 2 (Ac-HAVDIGGGCE).

Crosslinking

The hydrogel may be crosslinked by various known methods. In some embodiments, the hydrogel is crosslinked by enzymatic crosslinking, thermal crosslinking, a crosslinker, or a combination thereof.

In some embodiments, the hydrogel includes proteins or polypeptides, which may be crosslinked by a suitable enzyme catalyzing the formation of a chemical bond between proteins and polypeptides. For example, the crosslinking may be catalyzed by a transglutaminase, such as a microbial transglutaminase, which catalyzes the formation of isopeptide bonds between proteins. Suitable techniques for enzymatic crosslinking of protein-containing hydrogels include those described in O'Grady et al. (SLAS Technology, 2018, 23(6). 592-598), which is incorporated herein by reference in its entirety.

In some embodiments, the hydrogel may be crosslinked with a thermal free radical initiator. Suitable thermal initiators include azo-based radical initiators. Examples of this class of initiators include 2,2′-Azobis[2-(2-imidazolin-2-yl)propane]dihydrochloride (VA-044) and 2,2′-Azobis[2-methyl-N-(2-hydroxyethyl)propionamide] (VA-086). Suitable techniques for thermally crosslinking a hydrogels include those described in Zhen et al. (Brain Struct. Funct. 2016, 221(4), 2375-2383), which is incorporated herein by reference in its entirety.

In some embodiments, the hydrogels may be crosslinked by any suitable crosslinker that does not interfere with the function of the biomaterial to facilitate cell growth. The crosslinker may have at least one function group for attachment to the hydrogel and at least one crosslinkable group. In general, attachment of the crosslinker to the hydrogel provides a crosslinkable hydrogel, which may be crosslinked under suitable conditions. Various crosslinkers for making a crosslinkable hydrogel are known in the art. Suitable crosslinkers may include, for example, an UV-light activated crosslinker, a redox-activated crosslinker, a thermal polymerization initiator, or a combination thereof. Suitable crosslinkers may include those described in U.S. Pat. Nos. 5,686,504, 8,287,906, and WO 2019/055656, the entire contents of which are incorporated herein by reference.

Suitable UV-light activated crosslinkers include those having a vinyl group (—CH═CH₂). The vinyl group may be optionally substituted, for example, with an alkyl group. Examples of UV-light activated crosslinkers include alkyl acrylic acids, such as methacrylic acid, ethyl acrylate, n-propyl acrylate, isopropyl acrylate, n-butyl acrylate, isobutyl acrylate, n-amyl acrylate, iso-amyl acrylate, n-hexyl acrylate, isohexyl acrylate, cyclohexyl acrylate, isooctyl acrylate, 2-ethylhexy acrylate, decyl acrylate, lauryl acrylate, stearyl acrylate, or isobornyl acrylate. In particular embodiments, the hydrogel is crosslinked by methacrylic acid (HOOC—C(CH₃)═CH₂).

Suitable redox-activated crosslinkers include those having a phenol group (—C6H4OH). Examples of the crosslinkers having a phenol group include tyrosine (Tyr) and 3-(4-hydroxyphenyl)propionic acid (HPA). In some embodiments, the hydrogel with attached redox-activated crosslinkers are crosslinked by an oxidation reaction. In particular embodiments, a hydrogel with attached HPA is crosslinked by an oxidative coupling of HPA moieties catalyzed by hydrogen peroxide (H₂O₂) and horseradish peroxidase (HRP). Suitable techniques for crosslinking a hydrogel using redox-activated crosslinkers include those described in Wang et al. (Biomaterials, 2010, 31(6), 1148-1157), which is incorporated herein by reference in its entirety.

In particular embodiments, the hydrogel is porcine skin gelatin, the peptide is a SEQ ID No. 1, and the hydrogel is crosslinked by methacrylic acid. The resulting biomaterial may be referred to as “GelMA-Cad,” which includes methacrylated gelatin (GelMA, capable of being photopatterned) conjugated with a peptide from an extracellular epitope of N-cadherin.

In particular embodiments, the hydrogel is porcine skin gelatin, the peptide is a SEQ ID No. 2, and the hydrogel is crosslinked by 3-(4-hydroxyphenyl)propionic acid.

Preparation

In another aspect, provided is a method of preparing a biomaterial, comprising:

chemically attaching a peptide comprising a histidine-alanine-valine (HAV) sequence to a hydrogel; and

crosslinking the hydrogel having the attached peptide.

The hydrogel, peptide, and crosslinking processes are as described herein. In some embodiments, the hydrogel used for preparing the biomaterial comprise gelatin. In particular embodiments, the hydrogel used for preparing the biomaterial comprises porcine skin gelatin.

In some embodiments, the peptide used for preparing the biomaterial comprises SEQ ID NO: 1, SEQ ID NO: 2, or a variant thereof.

In some embodiment, the crosslinking process comprises enzymatic crosslinking, thermal crosslinking, chemically attaching a crosslinker to the hydrogel, or a combination thereof. Suitable regents and techniques for enzymatic crosslinking and thermal crosslinking processes, and suitable crosslinkers are as described herein. In some embodiments, the crosslinking comprises chemically attaching a crosslinker to the hydrogel; and crosslinking the hydrogel having the attached peptide and the attached crosslinker.

In some embodiments, a method of preparing a biomaterial is provided, which comprises:

chemically attaching a peptide comprising a histidine-alanine-valine (HAV) sequence to a hydrogel;

chemically attaching a crosslinker to the hydrogel; and

crosslinking the hydrogel having the attached peptide and the attached crosslinker.

In some embodiments, the peptide is chemically attached to the hydrogel prior to attaching the crosslinker to the hydrogel. In some embodiments, the crosslinker is chemically attached to the hydrogel prior to attaching the peptide. When the crosslinker is attached to the hydrogel prior to the attachment of the peptide, the peptide may be attached to the hydrogel at a position not occupied by the crosslinker, and/or to a crosslinker attached to the hydrogel.

In some embodiments, the crosslinker used for preparing the biomaterial includes a UV-light activated crosslinker, a redox-activated crosslinker, or a combination thereof. In some embodiments, the crosslinker used for preparing the biomaterial has an optionally substituted vinyl group, an optionally substituted phenol group, or a combination thereof.

In some embodiments, the crosslinker has a —C(CH₃)═CH₂ group. In particular embodiments, the crosslinker is methacrylic acid. In these embodiments, the crosslinking step may be initiated by UV light (such as a 25 mW/cm² UV light) in the presence of a photoinitiator. Examples of photoinitiators include lithium phenyl-2,4,6-trimethylbenzoylphosphinate (LAP). In some embodiments, the crosslinker is methacrylic acid, and the crosslinking step may initiate by exposing the hydrogel having the attached peptide and the attached crosslinker to photoinitiator LAP and UV light.

In some embodiments, the crosslinker is a UV-light activated crosslinker (e.g., one having a —C(CH₃)═CH₂ group), and the crosslinker is chemically attached to the hydrogel prior to the attachment of the peptide to the hydrogel. The subsequently attached peptide may be chemically attached to the hydrogel at a position not occupied by the crosslinker, and/or to a crosslinker attached to the hydrogel. In some embodiments, a method of preparing a biomaterial is provided, which comprises:

chemically attaching a UV-light activated crosslinker to a hydrogel to form a crosslinkable hydrogel;

chemically attaching a peptide comprising a histidine-alanine-valine (HAV) sequence to the crosslinkable hydrogel; and

exposing the resulting hydrogel to UV light, thereby causing the hydrogel to crosslink.

In some embodiments, the crosslinker is methacrylic acid, which is chemically attached to the hydrogel (such as gelatin) prior to the attachment of the peptide to the hydrogel. The subsequently attached peptide may be chemically attached to the hydrogel at a position not occupied by methacrylic acid, and/or to a methacrylic acid moiety attached to the hydrogel. In particular embodiments, a method of preparing a biomaterial is provided, which comprises:

chemically attaching methacrylic acid to a hydrogel to form a methacrylated hydrogel;

chemically attaching a peptide comprising a histidine-alanine-valine (HAV) sequence to the methacrylated hydrogel; and

exposing the resulting hydrogel to UV light, thereby causing the hydrogel to crosslink.

In some embodiments, the crosslinker is a redox-activated crosslinker. In some embodiments, the crosslinker is a redox-activated crosslinker having a phenol group, such as 3-(4-hydroxyphenyl)propionic acid. In these embodiments, one crosslinker may form covalent bond with another crosslinker under oxidative conditions, for example, horseradish peroxidase (HRP) and H₂O₂. In some embodiments, a method of preparing a biomaterial is provided, which comprises:

chemically attaching a peptide comprising a histidine-alanine-valine (HAV) sequence to form a functionalized hydrogel;

chemically attaching a redox-activated crosslinker to the functionalized hydrogel; and

subjecting the resulting hydrogel to an oxidation reaction, thereby causing the hydrogel to crosslink.

In particular embodiments, the crosslinker is 3-(4-hydroxyphenyl)propionic acid. A method of preparing a biomaterial is provided, which comprises:

chemically attaching a peptide comprising a histidine-alanine-valine (HAV) sequence to form a functionalized hydrogel;

chemically attaching 3-(4-hydroxyphenyl)propionic acid to the functionalized hydrogel; and

subjecting the resulting hydrogel to an oxidation reaction, thereby causing the hydrogel to crosslink.

In another aspect, the present disclosure provides a biomaterial produced by the preparation method disclosed herein. The produced biomaterial may be isolated or purified using known techniques before use.

Physical Properties

The biomaterial may have a stiffness of about 500 Pa to about 10 kPa. The stiffness may be at least 600 Pa, at least 800 Pa, at least 2 kPa, at least 4 kPa, at least 6 kPa, or at least 8 kPa. The stiffness may be less than 9 kPa, less than 7 kPa, less than 5 kPa, less than 3 kPa, or less than 1 kPa. In some embodiments, the biomaterial has a stiffness of about 800 Pa to about 5 kPa, such as about 1 kPa, about 2 kPa, about 3 kPa, or about 4 kPa. A desired stiffness may be achieved, for example, by changing the crosslinker (such as HPA) concentration. The crosslinker concentration may be varied by adjusting (1) the starting concentration of the crosslinker when conjugating to the hydrogel (such gelatin), and/or (2) the time allowed for conjugating to the hydrogel.

The biomaterial may have a pore size of about 10 μm to about 200 μm. The pore size may be at least 20 μm, at least 40 μm, at least 60 μm, at least 80 μm, at least 100 μm, at least 120 μm, at least 140 μm, at least 160 μm, or at least 180 μm. The pore size may be less than 190 μm, less than 170 μm, less than 150 μm, less than 130 μm, less than 110 μm, less than 90 μm, less than 70 μm, less than 50 μm, or less than 30 μm. In some embodiments, the biomaterial has a pore size of about 20 μm to about 80 μm, such as about 30 μm, about 50 μm, or about 70 μm.

### 3. Method

The biomaterial described herein, such as GelMA-Cad, may have physiological stiffness that can not only maintain photopatterned features, but additionally facilitate neuron (such as iPS C-derived glutamatergic neuron) survival and extension of neurite processes. Also, as compared to Matrigel, GelMA-Cad may support enhanced formation of synaptically connected neural networks, as measured by immunocytochemistry, electrophysiology, and viral synaptic tracing. Thus, the present biomaterials may aid the construction of three-dimensional neural tissue models to study human disease biology and augment drug screening assays. The present biomaterials may also facilitate vascular cell growth.

In one aspect, the present disclosure provides a method of contacting a plurality of cells with the biomaterial as described herein. In some embodiments, the plurality of cells may be derived from induced pluripotent stem cells (iPSCs), human pluripotent stem cells (hPSCs), tissue, mesenchymal stem cells, neural stem cells, or embryonic stem cells. The plurality of cells may be a neuron, a brain endothelial cell, a glial cell (e.g. oligodendrocytes, astrocytes, ependymal cells, Schwann cells, microglia, satellite cells), or a combination thereof.

iPSC-derived and hPSC-derived neurons are notoriously difficult to mature in two-dimensional and three-dimensional cultures without extended culture times or co-culture with astrocytes. It has been suggested that gelatin-based hydrogels can be neuroprotective and promote neurite outgrowth through integrin activation and integrin-dependent MAPK signaling. In some embodiments, the biomaterial as described herein may improve viability of the plurality of cells. In some embodiments, the viability of the plurality of cells may be at least about 88% after being embedded for about 2 days. In some embodiments, the viability of the plurality of cells may be at least about 95% after being embedded for about 3 days, about 4 days, about 5 days, about 6 days, about 7 days, about 8 days, about 9 days, or about 10 days. Surprisingly, the present method yields significantly more viable cells as compared to a gelatin-based hydrogel that does not comprise a cell adhesion molecule.

In some embodiments, the biomaterial as described herein may increase average neurite length of the plurality of cells. In some embodiments, the average neurite length of the plurality of cells may be about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, or about 71 μm after being embedded for about 3 days, about 4 days, about 5 days, about 6 days, about 7 days, about 8 days, about 9 days, or about 10 days.

In some embodiments, the biomaterial as described herein may increase average neurite width of the plurality of cells. In some embodiments, the average neurite width of the plurality of cells may be about 4 μm, about 5 μm, about 6 μm, or about 7 μm after being embedded for about 6 days, about 7 days, about 8 days, about 9 days, or about 10 days.

Typically, iPSC-derived and hPSC-derived neurons need to be cultured on two-dimensional monolayers of astrocytes to facilitate electrophysiological maturation (e.g. synaptogenesis). In some embodiments, the biomaterial as described herein may increase active synapses between the plurality of cells. In some embodiments, the active synapses between the plurality of cells may be at least about 80%, at least about 81%, at least about 82%, at least about 83%, at least about 84%, at least about 85%, at least about 86%, at least about 87%, at least about 88%, at least about 89%, at least about 90%, at least about 91%, or at least about 92% after being embedded for about 21 days. Surprisingly, the present method and biomaterial provides physical and biochemical cues and replaces the synaptogenic role of astrocytes when co-cultured with neurons. Further, remarkably, the present method yields a pronounced increase in the expression of postsynaptic terminal markers on neurons in the biomaterial as described herein relative to Matrigel alone.

In some embodiments, the plurality of cells may be differentiated into an organoid. The organoid may be a brain organoid, a gastrointestinal organoid, a lingual organoid, a thyroid organoid, a thymic organoid, a testicular organoid, a hepatic organoid, a pancreatic organoid, an epithelial organoid, a lung organoid, a kidney organoid, a gastruloid (embryonic organoid), a blastoid (blastocyst-like organoid), a cardiac organoid, or a retinal organoid.

Cortical organoids lack perfusable vasculature, cannot grow above a certain size before nutrient and oxygen transfer becomes diffusion-limited, do not exhibit appropriate laminar organization of distinct neuronal layers. The human cortex has well-defined cortical architectures, however cortical organoids have disorganized patterning with intermingled neurons. Because brain function is dependent on appropriately constructed neuronal circuits, and many diseases are due to faulty brain circuitry, this disorganization of neurons is limiting. In some embodiments, the organoid may be embedded in the biomaterial as described herein. The biomaterial as described herein may support organoid development that resembles human organs. For example, the biomaterial as described herein enables the brain organoid to be uniform and spherical. In another example, the brain organoid embedded in the biomaterial as described herein has laminar patterning of cortical layers. In some embodiments, the biomaterial may have perfusable channels that may be seeded with endothelial cells, mural cells, or combinations thereof. The perfusable channels seeded with cells may provide a functional vasculature throughout the organoid. The functional vasculature may increase size of the organoid, increase nutrient transfer and oxygen transfer to the organoid, and promote formation of distinct tissue layers as observed in human organs.

In another embodiment, tissue may be embedded in the biomaterial as described herein. The tissue may be mammalian tissue, fish tissue, reptilian tissue, bird tissue, amphibian tissue, or arthropod tissue. In another embodiment, the tissue may be human tissue or mouse tissue. In a further embodiment, the tissue may be brain tissue, lung tissue, stomach tissue, bladder tissue, liver tissue, kidney tissue, skin tissue, or any mammalian organ tissue known in the art. In another embodiment, the biomaterial as described herein may maintain vascular identity and promote angiogenesis in the brain endothelial cells. In another embodiment, the biomaterial as described herein may increase new blood vessel growth (e.g. angiogenesis) in a tissue. In some embodiments, the blood vessels may be an artery, a capillary, an arteriole, a venule, a vein, or a combination thereof. The blood vessels may comprise endothelial cells. The biomaterial as described herein may maintain vascular endothelial (VE)-cadherin expression in endothelial cells, a predominant feature of endothelial cells. Without being limited by any particular theory, it is hypothesized that the biomaterial as described herein mimics a heterotypic interaction that occurs between endothelial cells and mural cells, including vascular smooth muscle and pericytes. The biomaterial as described herein may support culture of the endothelial cells for standard applications, or in three-dimensional tissue assembly, or a combination thereof. In some embodiments, the endothelial cells may be non-brain endothelial cells.

A suitable density of the plurality of cells as described herein to be provided to the biomaterial may be at least about 0.1×10⁵ cells/cm², at least about 0.2×10⁵ cells/cm², at least about 0.3×10⁵ cells/cm², at least about 0.4×10⁵ cells/cm², at least about 0.5×10⁵ cells/cm², at least about 0.6×10⁵ cells/cm², at least about 0.7×10⁵ cells/cm², at least about 0.8×10⁵ cells/cm², at least about 0.9×10⁵ cells/cm², at least about 1×10⁵ cells/cm², at least about 1.1×10⁵ cells/cm², at least about 1.2×10⁵ cells/cm², at least about 1.3×10⁵ cells/cm², at least about 1.4×10⁵ cells/cm², at least about 1.5×10⁵ cells/cm², at least about 1.6×10⁵ cells/cm², at least about 1.7×10⁵ cells/cm², at least about 1.8×10⁵ cells/cm², at least about 1.9×10⁵ cells/cm², or at least about 2.0×10⁵ cells/cm².

### 4. Examples

Materials and Methods

Cell culture CC3 iPSCs were maintained in E8 medium on standard tissue culture plastic plates coated with growth-factor reduced Matrigel (VWR). At 60-70% confluency, the cells were passaged using Versene (Thermo Fisher) as described by Lippmann et al. (Stem Cells 2014, 32, 1032). Cortical glutamateric neurons were generated using a reported protocol (Shi et al., Nat. Protoc. 2012, 7, 1836) with some modifications. iPSCs were washed once with PBS and dissociated from the plates by incubation with Accutase (Thermo Fisher) for 3 minutes. After collection by centrifugation, cells were re-plated onto Matrigel-coated plates at a density of 2.5×10⁵ cells/cm² in E8 medium containing 10 μM Y27632 (Tocris). The following day, the medium was switched to E6 medium supplemented with 10 μM SB431542 (Tocris) and 0.4 μM LDN1931189 (Tocris) for 5 days to induce neuralization (Chambers et al., Nat. Biotechnol. 2009, 27, 275). Over the next 5 days, the media was gradually transitioned from E6 medium to N2 Medium (DMEM/F12 basal medium (Thermo Fisher) containing 1×N2 supplement (Gibco), 10 μM SB431542, and 0.4 μM LDN193189). On the 11th day of the differentiation, the resultant neural progenitors were dissociated by incubation with Accutase for 1 hour and passaged onto Matrigel in Neural Maintenance Medium with 10 μM Y27632 at a cell density of 1×105 cells/cm2. Neural Maintenance Medium was made by mixing a 1:1 ratio of N2 Medium and B27 Medium (Neurobasal Medium (Thermo Fisher) containing 200 mM Glutamax (Gibco) and 1×B27 (Gibco)). Cells received fresh Neural Maintenance Media every day for the next 20 days and a media change every 3-4 days afterwards. Neurons were used for experiments between days 70-100 of differentiation.

For the synaptic tracing experiments described below, a small population of neurons was also transduced with an adeno-associated virus (AAV) encoding EGFP under the control of the human synapsin promoter, which was a gift from Dr. Bryan Roth (Addgene plasmid #50465). Two weeks before the neurons were used, the cells were dissociated with Accutase and re-plated onto Matrigel-coated plates at a density of 2.5×10⁵ cells/cm² in Neural Maintenance Media containing 10 μM Y27632. The following day, the media was replaced, and the AAV was added at a MOI of 5,000. Fresh media was added to the cells after 24 hours in order to remove any residual virus and normal media changes were resumed thereafter.

GelMA synthesis and characterization Methacrylated gelatin (GelMA) was synthesized as described previously (Loessner et al., Nat. Protoc. 2016, 11, 727). Type A porcine skin gelatin (Sigma) was mixed at 10% (w/v) into DI water (sourced from an in-house Continental Modulab ModuPure reagent grade water system) at 60° C. and stirred until fully dissolved. Methacrylic acid (MA) (Sigma) was slowly added to the gelatin solution and stirred at 50° C. for 3 hours. The solution was then centrifuged at 3,500×g for 3 minutes and the supernatant was collected. Following a 5× dilution with additional warm (40° C.) UltraPure water (Thermo Fisher) to stop the reaction, the mixture was dialyzed against DI water for 1 week at 37° C. using 12-14 kDa cutoff dialysis tubing (Fisher) to remove salts and MA. The pH of the solution was then adjusted to 7.35-7.45 by adding HCl or NaOH as measured with a Thermo Fisher Scientific Orion Star pH meter. The resulting GelMA solution was lyophilized for 3 days using a Labconco lyophilizer and stored at −20° C.

Peptide conjugation and characterization Peptides were conjugated to GelMA as previously reported (Bian et al., Proc. Natl. Acad. Sci. 2013, 110, 10117) with slight modifications. Briefly, GelMA was reconstituted in triethanolamine (TEOA) buffer (Sigma) to create a 10% w/v solution and stirred at 37° C. for 2 hours until fully dissolved. The pH of the solution was then adjusted to 8.0-8.5 using HCl or NaOH. Scrambled (Ac-AGVGDHIGC, to make GelMA-Scram) or N-Cadherin mimic (Ac-HAVDIGGGC, to make GelMA-Cad) peptides (GenScript) were added to the GelMA/TEOA buffer to form a 1% w/v solution. The cysteine residue at the C-terminal end of the peptides permitted a Michael-type addition reaction with GelMA. The solution was stirred at 37° C. for 24 hours and then dialyzed against DI water using 6-8 kDa cutoff dialysis tubing (Spectrum) for 1 week at 37° C. The pH of the solution was then adjusted to 7.35-7.45 using HCl or NaOH, and the solution was lyophilized and stored at −20° C. Conjugation was routinely verified through 1H-NMR using a Bruker 500 Hz NMR spectrometer set to 37° C. for the presence of the amino acid valine.

An alternative process for conjugating a peptide to the gelatin backbone of GelMA may be used as follows: GelMA is reconstituted in triethanolamine buffer to create a 10% solution, and stirred at 37° C. for 2 hours until fully dissolved. The pH is adjusted between 8-8.5. The peptide is then added to the hydrogel (between 0.1%-5% weight/volume), and the mixture is stirred at 37° C. for 24 hours. The solution is then filtered and dialyzed using a tangential flow filtration system (2 kDa pore size).

Fourier-transform infrared spectroscopy 198 mg of potassium bromide (Sigma) was added to 2 mg of lyophilized gelatin, GelMA, GelMACad, or GelMA-Scram and crushed using a mortar and pestle. The crushed samples were transferred to a 13 mm Specac evacuable pellet press die and compressed into a thin disc using a Specac manual hydraulic press. An additional disc was made using only potassium bromide for calibration. Samples were stored in a dry container overnight and analyzed the following day using a Bruker Tensor 27.

Atomic force microscopy GelMA, GelMA-Scram, and GelMA-Cad were reconstituted and polymerized into hydrogel discs as described in the cell seeding section herein. A Bruker Dimension Icon Atomic Force Microscope was used to measure hydrogel stiffness. 0.01 N/m Novascan probes containing a 4.5 μm polystyrene bead (PT.PS.SN.4.5.CAL) were used to measure three distinct 5×5 μm areas of each hydrogel. Three hydrogel disc replicates of each sample were included for a total of 576 stiffness measurements per sample. For each individual force curve, a first order baseline correction was performed, and the Hertzian model was used to calculate Young's modulus. For tool calibration, polyacrylamide hydrogels were prepared as previously reported (Stroka, et al., Blood 2011, 118, 1632) and measured prior to GelMA and its derivatives.

Scanning electron microscopy Lyophilized GelMA, GelMA-Cad, and GelMA-Scram were reconstituted in PBS to form 10% (w/v) solutions with 0.05% LAP initiator (Sigma). 30 μL of each hydrogel solution was added to a Ted Pella pin mount and crosslinked by an 8 second exposure to a 25 mW/cm² UV light using a ThorLabs UV Curing LED System. These pin mounts were stored in a Ted Pella mount storage tube and then placed in a −80° C. freezer overnight. The following day, the samples were transferred to a Labconco lyophilizer for an additional 2 days and then stored at room temperature until used. To characterize the internal microscructures of GelMA, GelMA-Cad and GelMA-Scram, the dried samples were observed using a scanning electron microscope (Zeiss Merlin) at an accelerating voltage of 2 kV. ImageJ software was used to quantify pore sizes, where the mean diameter of each pore was considered the average pore size.

Fabrication and seeding of hydrogel scaffolds GelMA, GelMA-Scram and GelMA-Cad were reconstituted in Neuron Maintenance Media to make a 10% (w/v) solution with 0.05% LAP initiator. iPSC-derived neurons were detached from 12-well plates via a 5 minute incubation with Accutase and centrifuged for collection. Unless otherwise stated, neurons were mixed with reconstituted hydrogel/initiator solution to achieve a density of 2×10⁵ cells/mL. For some experiments, GelMA was mixed with soluble peptide rather than via covalent coupling; here, soluble peptides were reconstituted in DMSO to create a 10 mg/mL solution, and then the peptides were added to the GelMA/initiator/neuron solution to achieve a 50 μg/mL peptide concentration. Once the solutions were prepared, they were mixed thoroughly with a P1000 pipette to break up any cell clumps. Next, 100 μL of the cell suspension was added to RainX-treated glass slides and covered with 12 mm diameter coverslips (Carolina) to form discs. These discs were then exposed to 25 mW/cm² UV light for 8 seconds and set aside for 10 minutes at room temperature. Hydrogel discs were then removed from the glass slides and transferred to a 12-well plate with 1 mL of Neural Maintenance Media per well.

To embed neurons in Matrigel, 1 mL Matrigel aliquots were thawed on ice. Once thawed, the neurons were embedded at the same cell density as described above, and 100 μL of the solution was added directly onto the coverslips in a 12-well plate. The plate containing the Matrigel discs was placed in an incubator at 37° C. to crosslink for 30 minutes. After the Matrigel was fully crosslinked, 1 mL of Neural Maintenance Media was added to each of the wells. For all conditions, media was replaced twice a week until cells were used.

Live/dead cell imaging To assess long term cell viability, hydrogel discs were incubated with CytoCalcein™ Violet 450 (AAT Bioquest) and propidium iodide (PI, Thermo Fisher) for one hour. The hydrogel discs were imaged using a Zeiss 710 confocal microscope and cell viability was quantified using ImageJ. Following imaging, 1 mL of Neural Maintenance Media was added to each well in order to dilute any remaining Calcein/PI from the hydrogels.

Neurite projection quantification Raw data were exported in 16-bit TIF format and imported into Matlab 2017 for quantification using a custom image analysis script. Briefly, images were smoothed using a 3×3 pixel smoothing filter to mitigate image noise, and in-focus neurite segments were identified by isolating regions at least 5% brighter than the mean pixel intensity in the surrounding 50-pixel radius. Cell bodies and neurites were distinguished by successive erosion of the resulting binary mass. The erosion radius at which the total cell mass declined most steeply was used to define the radius required to erode neurites while sparing cell bodies. Following segmentation of neurites and cell bodies, algorithms previously developed for analysis of mitochondrial networks (McClatchey et al., Mitochondrion 2016, 26, 58) were used to measure the average length and width of each neurite segment.

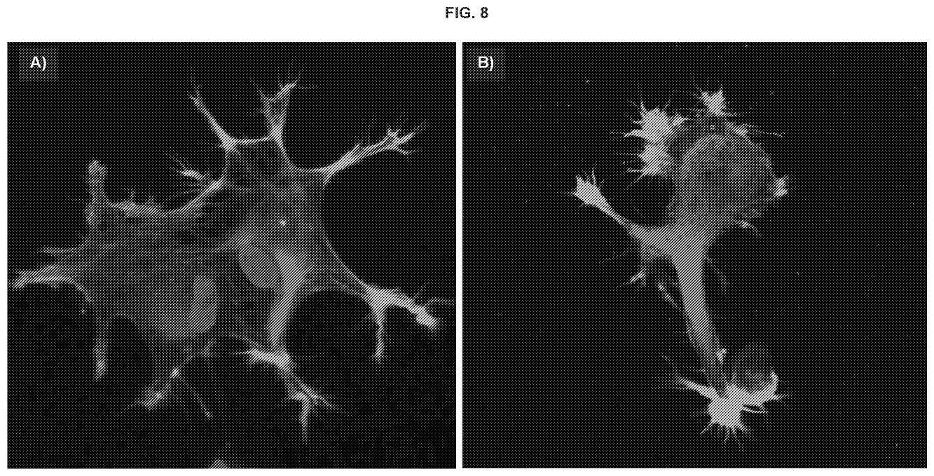

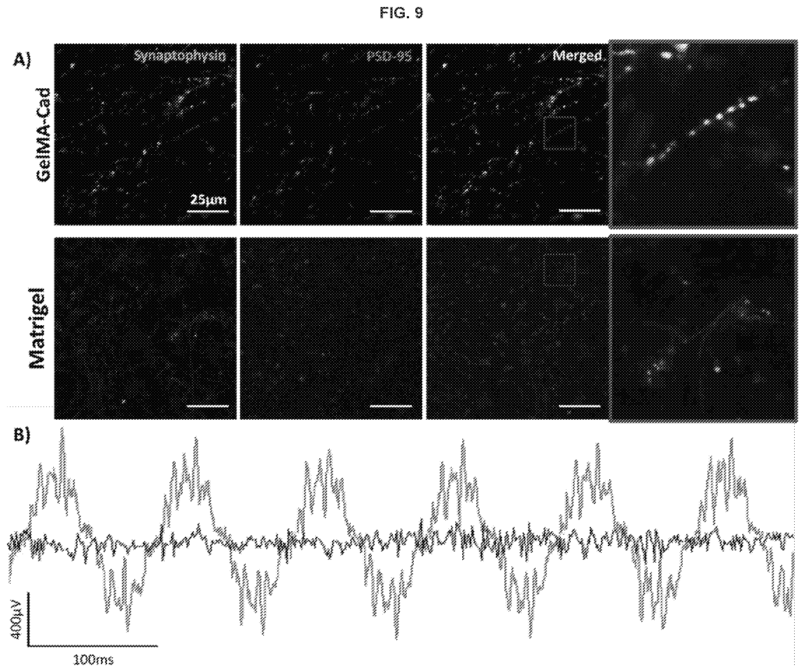

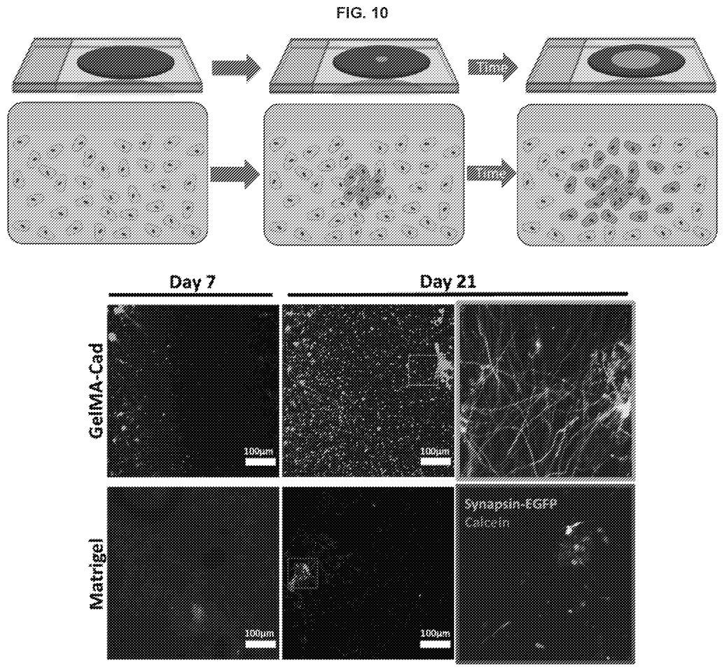

Synaptic tracing Hydrogel discs were fabricated as described above. Prior to crosslinking (of GelMA-Cad) or gelation (of Matrigel), neurons transduced with synapsin-driven EGFP were dissociated from plates via a 5 minute incubation with Accutase and then added to the center of the hydrogel disc at a density of 2×10³ cells/mL (as shown in FIG. 10). After crosslinking or gelation, the hydrogel discs were placed in 1 mL of Neural Maintenance Media and stored in an incubator at 37° C. until imaged. For all conditions, the media was replaced twice a week. The formation of synaptic connections was visualized by the spread of EGFP fluorescence across each hydrogel using a Zeiss LSM 710 confocal microscope.

Immunofluorescence After 2 weeks of culture, neurons embedded in hydrogels were fixed in 4% PFA (Sigma) for 20 minutes and then washed 3 times with PBS. A solution of 5% goat serum and 0.03% Triton X-100 (Thermo Fisher) was then added to the hydrogels overnight on a rocking platform at room temperature. The hydrogels were then incubated overnight with DAPI and a combination of the following fluorescently conjugated primary antibodies: bIII tubulin Alexa Fluor 647 (Abcam ab190575), PSD-95 Alexa Fluor 488 (Novus Biologicals NB300556AF488), and/or synaptophysin Alexa Fluor 555 (Abcam ab206870). Hydrogels were then imaged using a 40× objective on a Zeiss LSM 710 confocal microscope. The number of PSD-95 and synaptophysin puncta was quantified using the cell counter plugin on ImageJ. Colocalization of these two markers was quantified using Zeiss Zen Black software.

Electrophysiology Neurons embedded in GelMA-Cad or Matrigel hydrogels were recorded in a bath consisting of 140 mM NaCl, 2.8 mM KCl, 2 mM CaCl₂, 2 mM MgCl₂, 10 mM HEPES, and 10 mM D-glucose. Sharp glass microelectrodes were prepared from borosilicate glass with a Sutter P97 pipette puller and filled with extracellular solution to reach a resistance of 6-8 MΩ. The recording electrode was placed near the edge of the hydrogel disc. Whole-cell patch clamp recordings were performed in a recording chamber placed on the stage of a Zeiss Axioscope upright microscope. Current clamp experiments were performed with an Axon Multiclamp 700A amplifier. Data recording and analysis were performed with Axon pClamp software.

### Example 1. Synthesis and Characterization of GelMA Functionalized with N-Cadherin Peptide

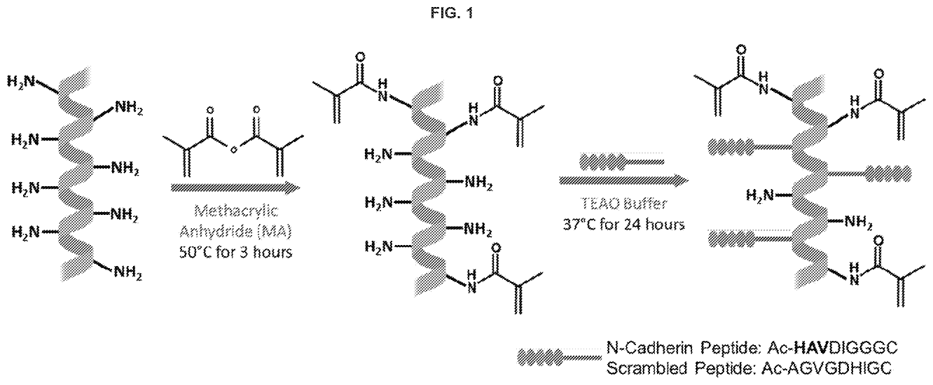

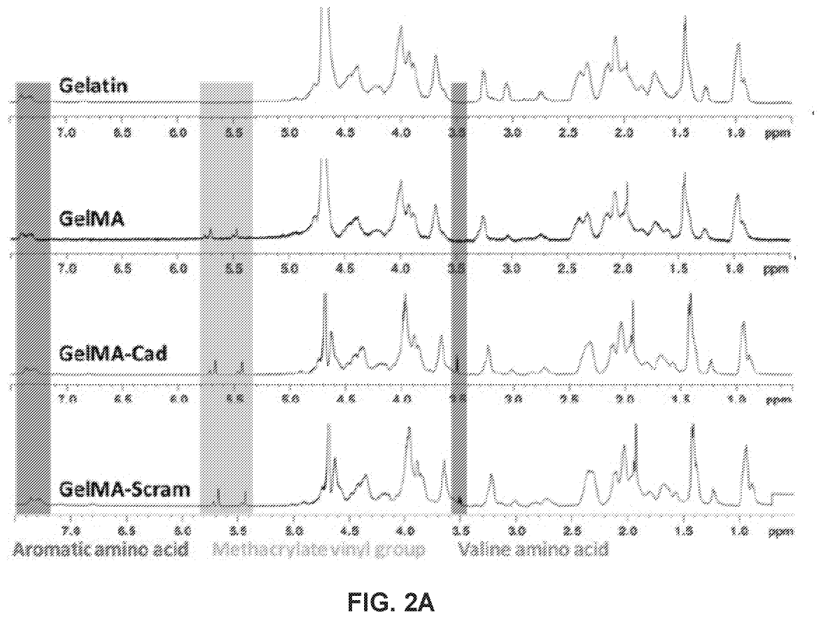

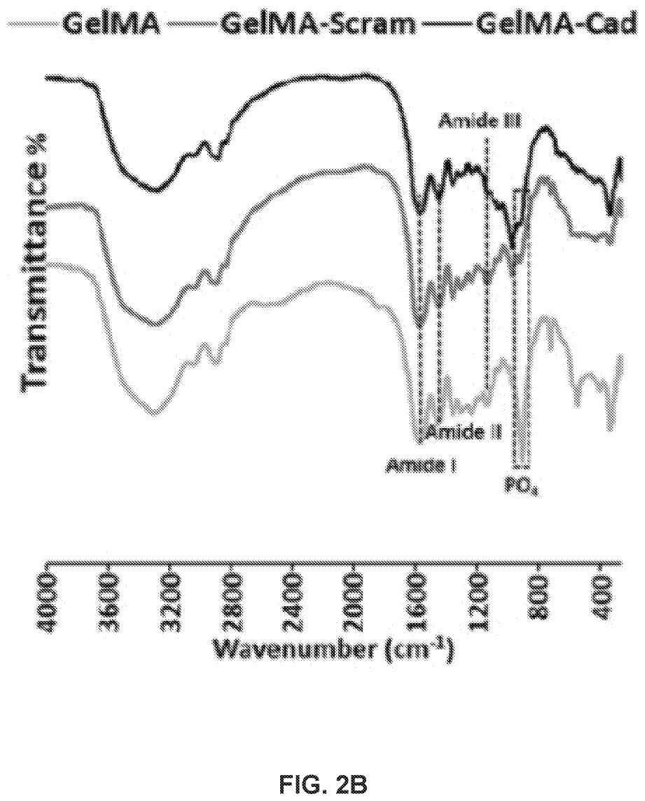

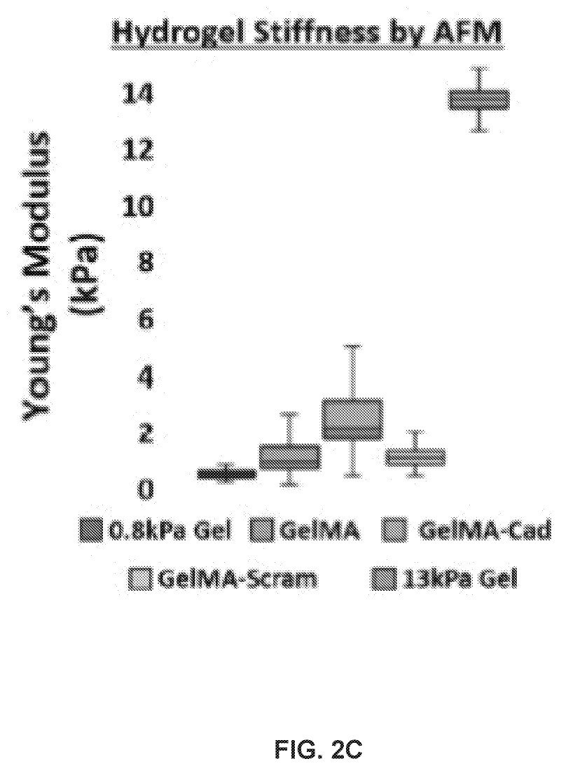

GelMA was chosen as a base material due to its ease of handling and robust mechanical properties (after crosslinking) compared to ECMs such as Matrigel and HA. N-cadherin functionality was chosen for the role of this cell adhesion molecule in neurite growth during neurogenesis. The extracellular peptide epitope of N-cadherin chosen for this study has previously been used to functionalize methacrylated HA in order to support chondrogenesis from mesenchymal stem cells, but 3D scaffolds fabricated with this peptide have not been used to support neural cultures. To generate the GelMA-Cad scaffold, porcine gelatin was first functionalized with methacrylic anhydride in order to create the GelMA backbone that could be crosslinked when exposed to the photoinitiator LAP and UV light (FIG. 1). This modification was confirmed through the presence of methacrylic side chain protons (˜5.45 and 5.7 ppm) using ¹H-NMR (FIG. 2A). GelMA was then functionalized with the extracellular epitope of N-cadherin (HAVDIGGGC) to prepare GelMA-Cad, or with an N-cadherin-scrambled peptide (AGVGDHIGC) to prepare GelMAScram. The conjugation of these peptides to the scaffold was also confirmed via ¹H NMR through the presence of valine protons (˜3.5 ppm), which are not present in the gelatin or GelMA spectra (FIG. 2A). Additionally, Fourier-transform infrared spectroscopy (FTIR) was employed to further validate successful functionalization. The FTIR transmittance spectra showed a noticeable decrease in PO₄ peaks (1000 cm⁻¹) and amide peaks I, II, III (1640, 1540, and 1250 cm⁻¹, respectively) in GelMA-Cad and GelMA-Scram samples compared to GelMA (FIG. 2B), likely due to peptide conjugation. Collectively, these data suggest GelMA was properly synthesized and functionalized.

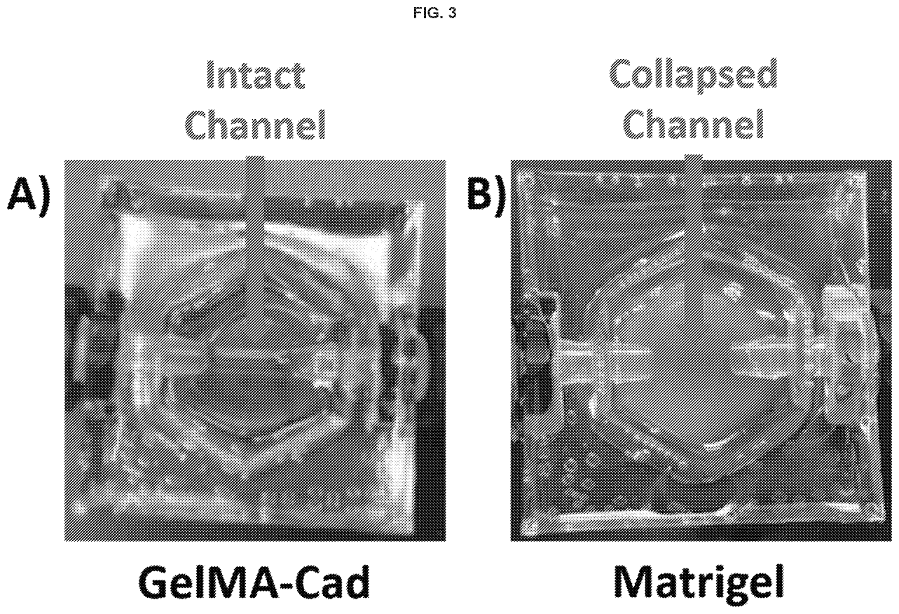

The mechanical and physical properties of the crosslinked hydrogels were studied. In order to determine the stiffness of GelMA, GelMA-Cad, and GelMA-Scram, atomic force microscopy (AFM) was performed. 0.8 kPa and 13 kPa polyacrylamide hydrogels were produced and measured by AFM to validate that the tool was properly calibrated (FIG. 2C). After crosslinking with LAP and UV light, GelMA, GelMA-Cad, and GelMA-Scram exhibited stiffness values of approximately 1-5 kPa (FIG. 2C), which resembles the stiffness of native brain tissue. Despite its relatively low elastic modulus, GelMA-Cad is stiff enough to maintain patterned architectures: when it was crosslinked around silicone tubing, followed by manual extraction of the tubing, a straight, a perfusable channel remained in the GelMA-Cad (FIG. 3A), whereas Matrigel collapses and the perfusion channel does not remain patent (FIG. 3B). Thus, similar to GelMA, GelMA-Cad can be patterned into more complex structures.

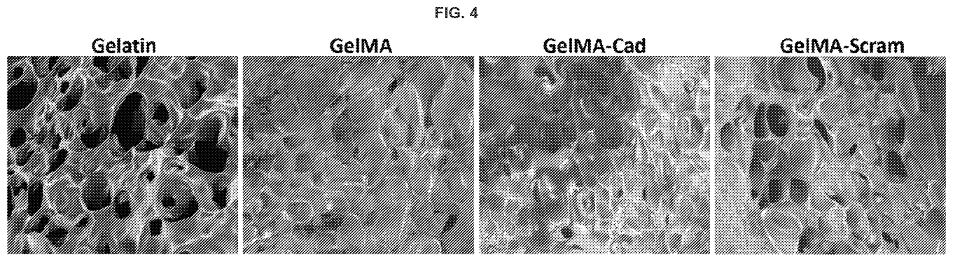

The microstructure of the hydrogels was characterized by scanning electron microscopy (SEM). Porous network structures are commonly observed in hydrogels and are important for nutrient diffusion, cell integration and removal of waste products, and the degree of chemical substitution has an inverse relation to pore size upon crosslinking. The average pore size diameter of GelMA, GelMA-Cad, and GelMA-Scram were measured at 42.8±0.2, 43.1±0.2, and 42.4±0.2 μm, respectively (FIG. 4). These measurements confirm that the hydrogels all have similar physical and mechanical properties, such that differences in neuron behavior can likely be attributed to bioinstructive cues.

### Example 2. GelMA-Cad Hydrogels Support Survival and Outgrowth of iPSC-Derived Neurons

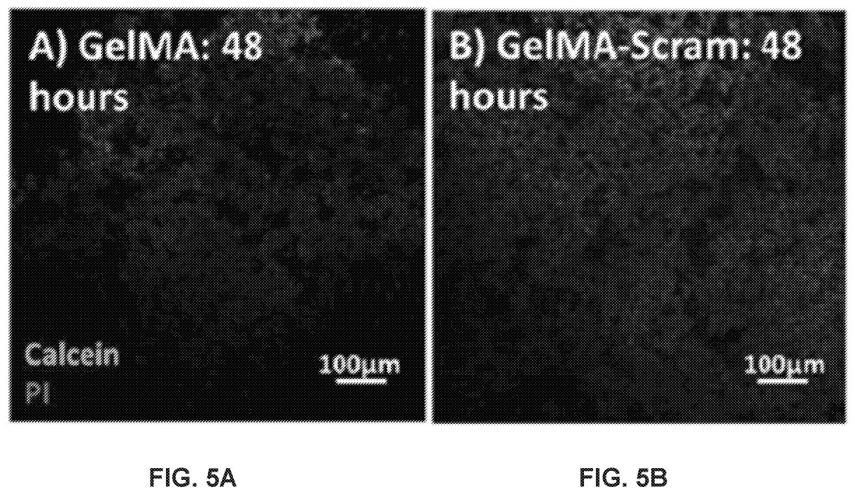

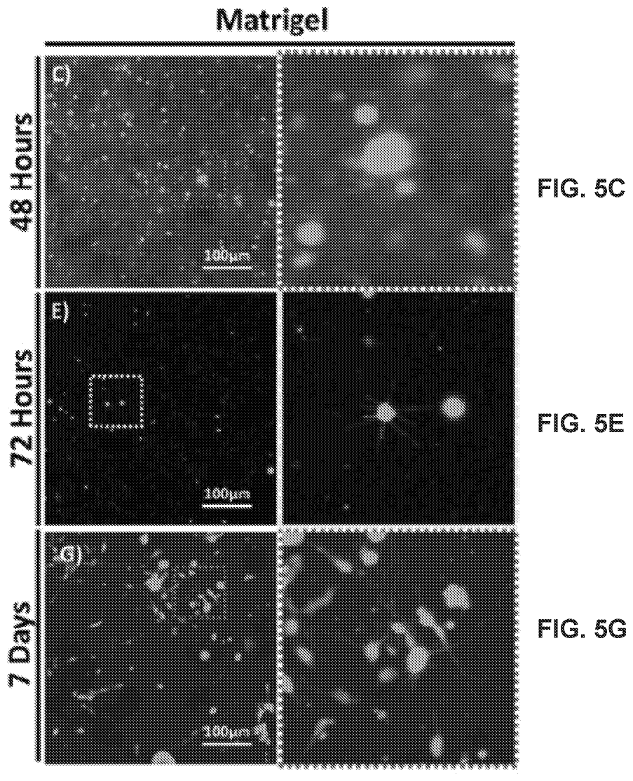

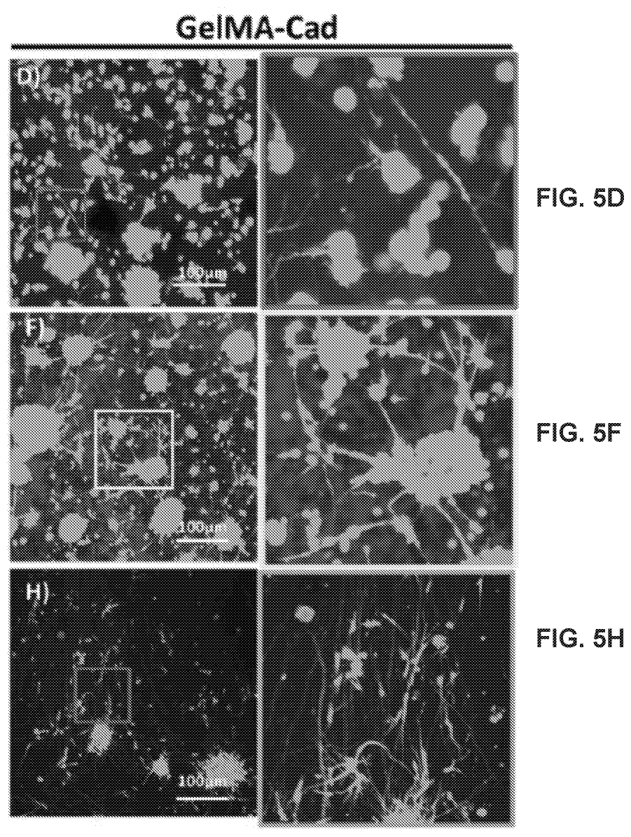

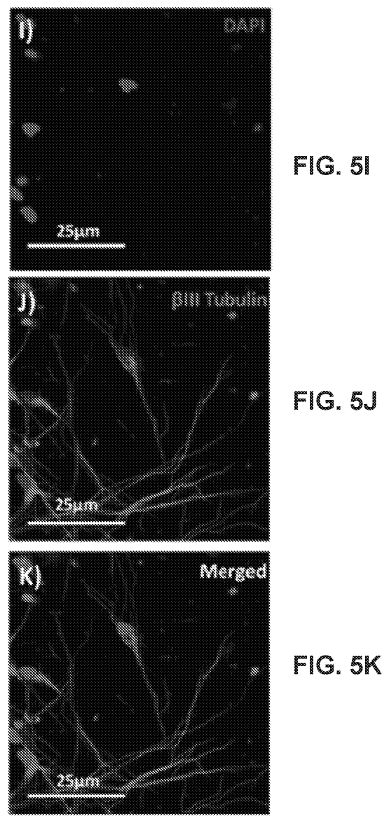

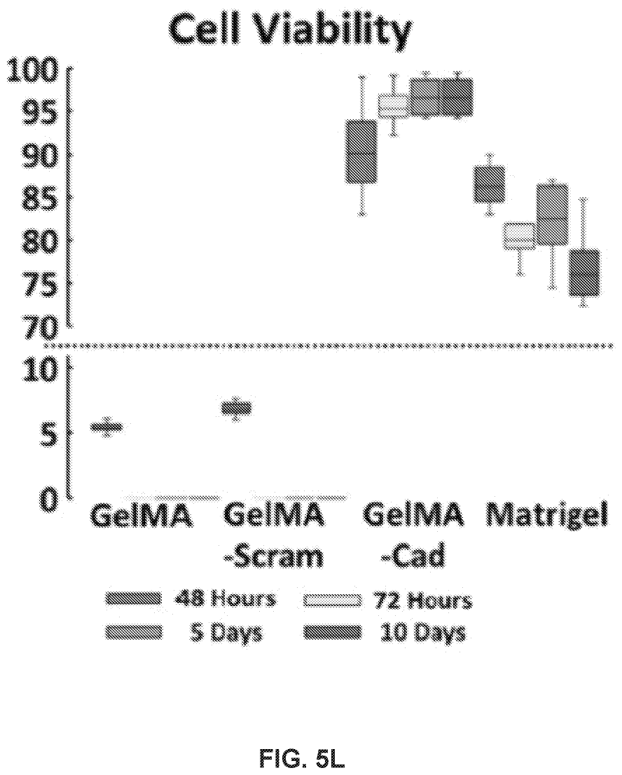

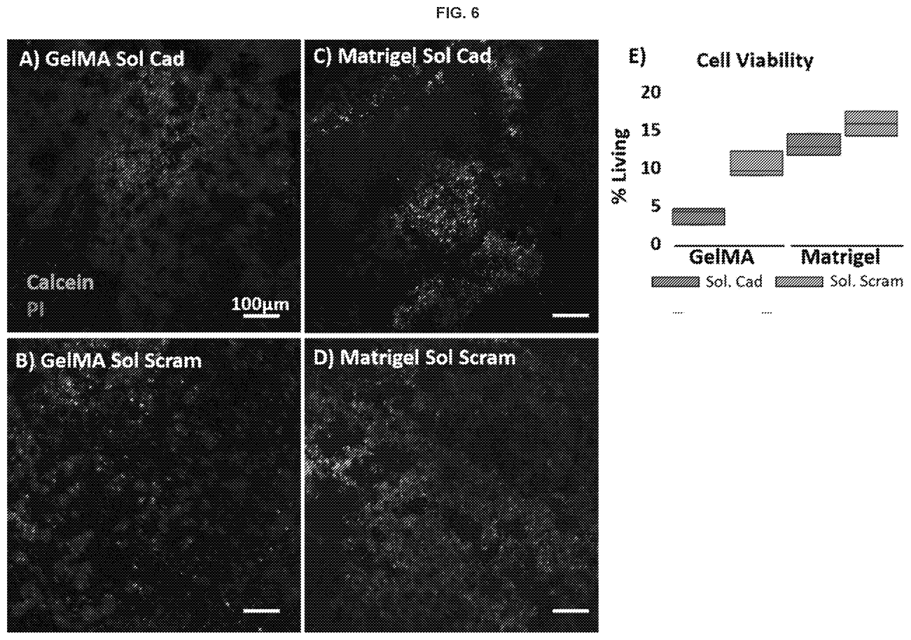

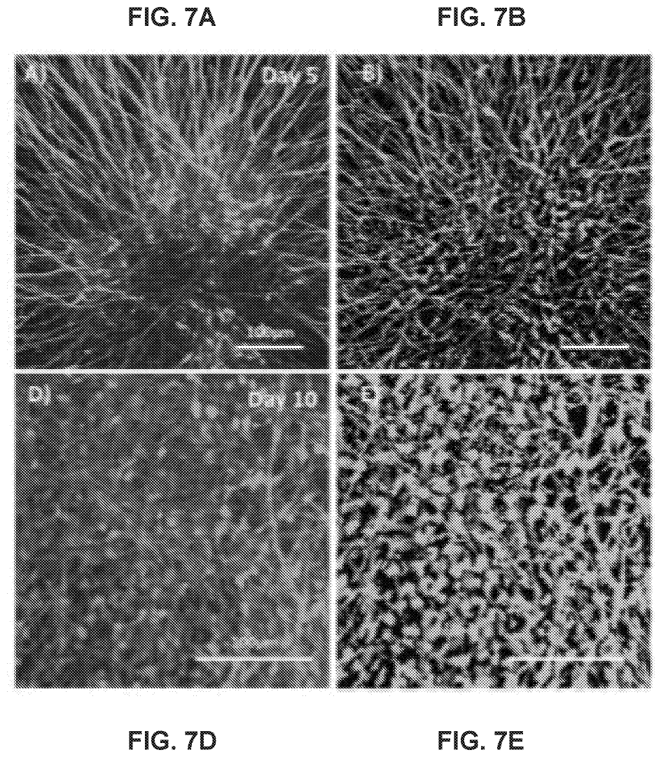

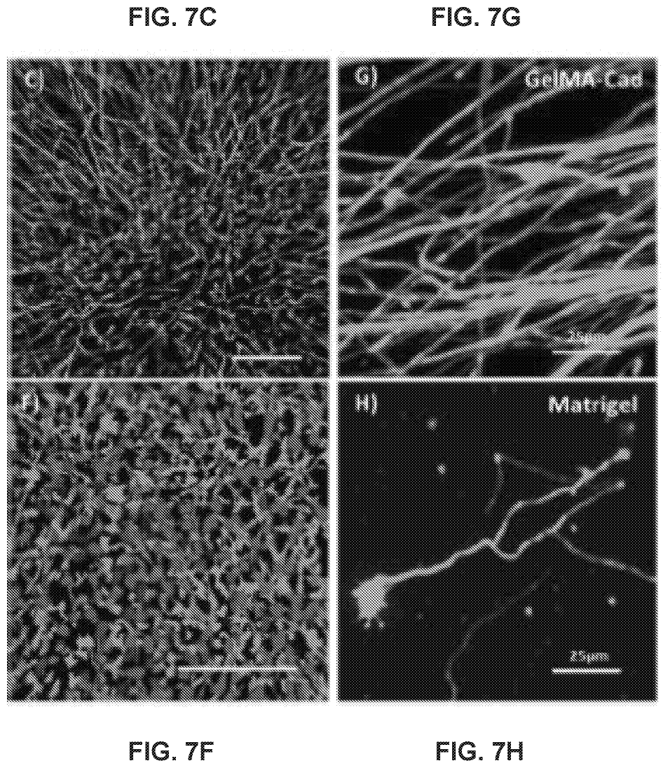

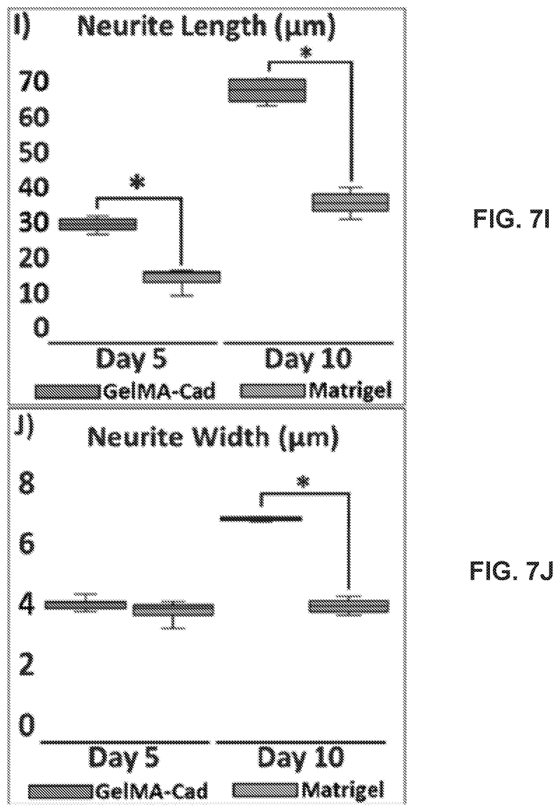

To assess the ability of hydrogels to support human neuron survival and outgrowth, human iPSCs were differentiated into cortical glutamatergic neurons and cultured for 70-100 days before use. These neurons were then dissociated into single-cell suspensions and embedded into Matrigel, GelMA-Cad, GelMA-Scram, or GelMA. As a negative control for physical conjugation of peptides to the hydrogels, neurons were also embedded in GelMA with either soluble N-cadherin peptide or soluble scrambled peptide. Using calcein and propidium iodide dyes to mark live and dead cells, respectively, we determined that neurons embedded in GelMA and GelMA-Scram (both conjugated and soluble peptide), as well as Matrigel with the soluble peptide, died within 4 days (FIG. 5 and FIG. 6). Meanwhile, neurons in conjugated GelMA-Cad and Matrigel exhibited viability of 90.2±1.3% and 86.3±2.2% after 2 days, respectively. After 3 days, neurons in conjugated GelMA-Cad exhibited viability of 96.7±1.2% while viability in Matrigel decreased slightly to 80±1.3%. After 5 days, viability remained relatively constant (96.7±1.1% in conjugated GelMA-Cad versus 82.3±1.9% in Matrigel). By day 10, viability in conjugated GelMA-Cad continued to remain constant at 96.7±1.6% whereas viability in Matrigel again decreased slightly to 76.7±0.8%. Next, we monitored neurite projections from neurons embedded in either Matrigel or conjugated GelMA-Cad (referred to solely as GelMA-Cad from hereon) after 5 and 10 days using calcein. Neurite length and width are frequently employed as measures of neuron health and connectivity, and so we quantified Z-stack images of neurites using a custom Matlab script (FIGS. 7A-7H). On day 5, relative to Matrigel, neurons embedded in GelMA-Cad exhibited significantly higher average neurite length (28.9±1.6 μm vs 14.1±2.6 μm; p<0.05), whereas average neurite width was not significantly different between GelMA-Cad and Matrigel (4.0±0.2 μm vs 3.7±0.2 μm) (FIGS. 71-7J). However, on day 10, relative to Matrigel, neurons in GelMA-Cad exhibited significantly higher average neurite length (67.2±3.2 μm vs 35.3±7.1 μm; p<0.05) and average neurite width (6.8±0.2 μm vs 3.9±0.2 μm; p<0.05) (FIGS. 71-7J). These results demonstrate GelMA-Cad is an effective hydrogel for enhancing survival and maturation of human iPSC-derived neurons by morphometric parameters.

### Example 3. GelMA-Cad Hydrogels Support Outgrowth and Functionality of iPSC-Derived Astrocytes

To assess the ability of hydrogels to support human astrocyte outgrowth and functionality, human iPSCs were differentiated into astrocytes and cultured for 30 days before use. These astrocytes were then dissociated into single-cell suspensions and embedded into GelMA-Cad. As a positive control for astrocyte activation, astrocytes were also embedded in GelMA-Cad with TNF-alpha. To study outgrowth, health and functionality/activation of the astrocytes, GFAP (red), actin (green), and DAPI nuclear stain (blue) were used (FIG. 8). Astrocytes in GelMA-Cad (FIG. 8A) extend their processes and have minimal GFAP expression, indicating quiescence and maturity. Astrocytes in GelMA-Cad treated with TNF-alpha to activate inflammation have an upregulation in GFAP, indicating that the astrocytes respond appropriately to inflammation (FIG. 8B). These results demonstrate that GelMA-Cad is an effective hydrogel for enhancing survival, maturation, and function of human iPSC-derived astrocytes.

### Example 4. iPSC-Derived Neurons Form Synaptically Connected Networks in GelMA-Cad Hydrogels

The increased length and diameter of neurons in GelMA-Cad suggests improved functional properties, which we sought to validate with additional metrics including immunostaining, electrophysiological recordings, and viral synaptic tracing. First, embedded neurons were fixed and immunostained for synaptophysin (a presynaptic terminal marker) and PSD-95 (a postsynaptic terminal marker). Neurons embedded in GelMA-Cad expressed both markers 21 days after embedding (average of 492 synaptophysin puncta and 423 PSD-95 puncta per 75 μm3), and there was an average of 87.3±1.3% colocalization, which indicates the formation of an active synapse (FIG. 9A). Neurons embedded in Matrigel had substantially lower expression of synaptophysin and PSD-95 (average of 82 puncta and 28 puncta per 75 μm3, respectively), with only 13.3±3.3% colocalization of the presynaptic and postsynaptic markers (FIG. 9A), indicating a substantially lower number of prospective synapses. Next, to assess synaptic connectivity, electrical activity of the embedded neurons were measured through patch clamping. Action potentials were readily measured within neurons embedded in GelMA-Cad (FIG. 9B, red line), but only minimal activity was observed in Matrigel-embedded neurons (FIG. 9B, black line), thus providing evidence that the N-cadherin peptide improves functional maturity.

To assess widespread neural network formation, synaptic tracing experiments were conducted by transducing iPSC-derived neurons with an adeno-associated virus (AAV) encoding EGFP under the control of human synapsin promoter (where synapsin is a presynaptic terminal marker). Wild-type neurons were mixed with hydrogel precursor, and prior to crosslinking the hydrogels, a small population of AAV-transduced neurons (1:100 ratio of transduced to non-transduced neurons) were injected into the center (FIG. 10). The spread of the EGFP signal could thus be monitored over time to elucidate the degree of neural network formation across the hydrogel. Limited EGFP spread was observed after 7 days in both hydrogels, which is consistent with FIG. 7 demonstrating that neurite length and width are still increasing at this early time point. However, after 21 days, EGFP had propagated to virtually every neuron within the GelMA-Cad hydrogels, whereas sparse EGFP spread was observed in Matrigel (FIG. 10). Calcein dye was added to each hydrogel to show that the neurons in Matrigel were alive but not synaptically connected. Therefore, only neurons in the GelMA-Cad hydrogels were able to propagate the virus through functional synapses across the entire tissue construct. Overall, these data strongly suggest that GelMACad facilitates the maturation of iPSC-derived neurons on a functional level.

### Example 5. GelMA-Cad Hydrogels Prevent iPSC-Derived Brain Endothelial Cells (BMECs) from De-Differentiating and Losing their Vascular Phenotype

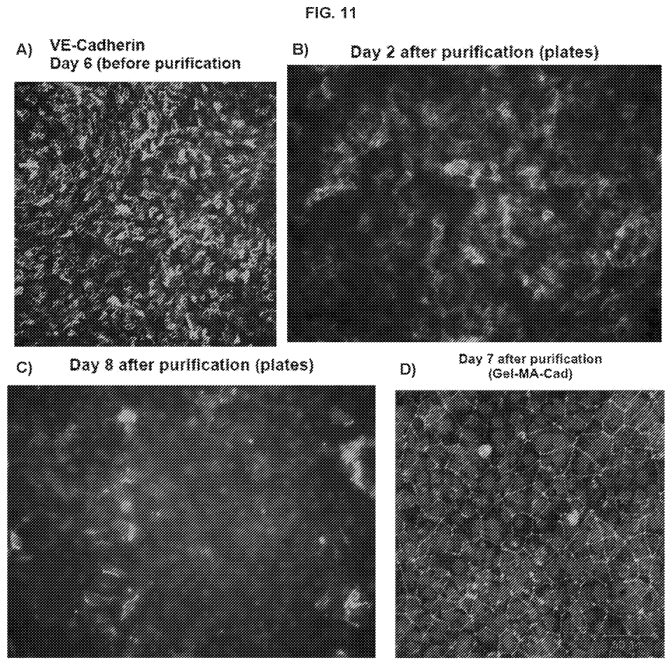

To assess the ability of hydrogels to prevent iPSC-derived BMECs from de-differentiating and losing their vascular phenotype, iPSCs were differentiated into BMECs according to established protocols (FIG. 11A). Then, the BMECs were purified for extended culture on plastic dishes with or without GelMA-Cad (FIGS. 11B-11D). Maintenance of VE-cadherin expression in cell junctions is indicative of BMEC vascular phenotype. Using a VE-cadherin stain (green) and DAPI nuclear stain (blue) demonstrated that GelMA-Cad maintains and supports formation of junctions between BMECs, thus maintaining their cellular phenotype (FIG. 11D).

### Example 6. GelMA-Cad Hydrogels Support Vascular Growth in Primary Brain Tissue

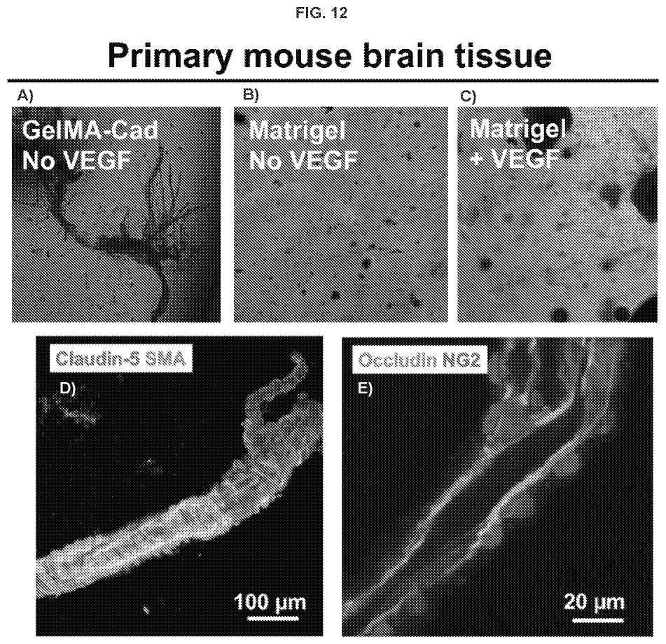

To determine whether hydrogels support vascular growth in primary brain tissue, mouse hippocampus and cortex were dissected and embedded in GelMA-Cad, Matrigel, or Matrigel with vascular endothelial growth factor (VEGF). Brightfield images show that new vessels only sprout in GelMA-Cad hydrogels and not Matrigel hydrogels (FIGS. 12A-12C). Sprouted vessels include arteries and capillaries consisting of a single lumen. Arteries are larger vessels with multiple claudin-5-positive endothelial cells lined by smooth muscle actin (SMA)-positive smooth muscle (FIG. 12D). Capillaries are smaller vessels with occludin-positive endothelial cells lined with a single layer of neuron-glial antigen 2 (NG2)-positive pericytes (FIG. 12E). These data show that GelMA-Cad hydrogels support vascular growth in primary brain tissue, whereas Matrigel hydrogels do not, even when provided with a vascular growth factor.

### Example 7. GelMA-Cad Hydrogels Support Complex Structure Formation in Brain Organoids Differentiated from iPSCs

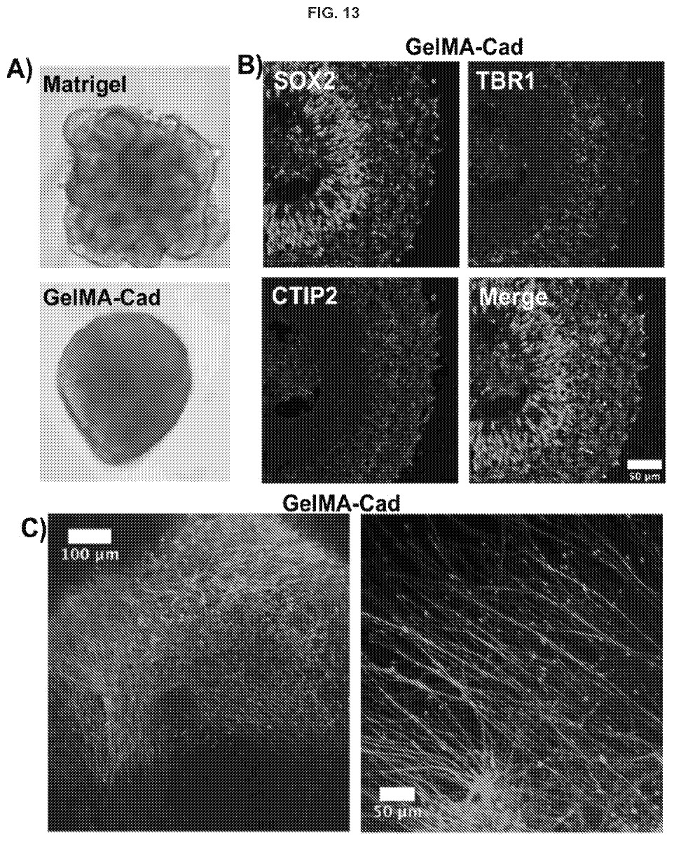

To assess the ability of hydrogels to support complex structure formation in 3D brain organoids, human iPSCs were differentiated into brain organoids. These organoids were embedded into Matrigel or GelMA-Cad. A brightfield image of a brain organoid embedded in Matrigel and a brightfield image of a brain organoid embedded in GelMA-Cad revealed that brain organoids embedded in GelMA-Cad show more uniform spherical compaction and no disorganized neuroepithelial buds as compared to brain organoids embedded in Matrigel (FIG. 13A). Using SOX2, TBR1, CTIP2 staining to mark laminae, it was determined that brain organoids embedded in GelMA-Cad exhibited laminar patterning of deep cortical layers as marked by distinct regions of TBR1 and CTIP2 (FIG. 13B). Further, brain organoids embedded in GelMA-Cad exhibit robust neuronal outgrowth (FIG. 13C). Therefore, these results demonstrate that GelMA-Cad facilitates complex structure formation in brain organoids.

### Example 8. Preparation and Use of Redox-Crosslinking Hydrogel

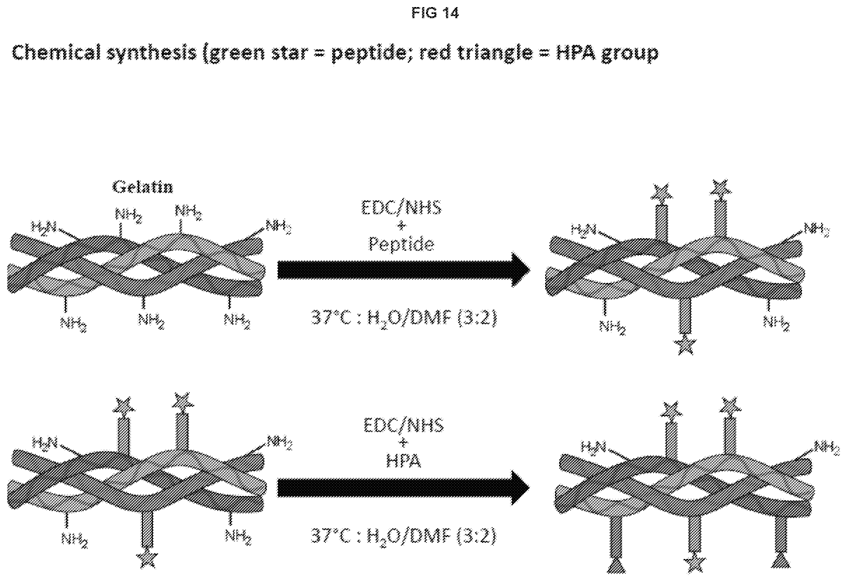

The following process was carried out for conjugating peptide to the gelatin backbone and crosslinking the hydrogel using redox activated crosslinker (redox gel, FIG. 14). Gelatin was reconstituted to a 4-10% solution in PBS. The solution is stirred at 50° C. until dissolved. To activate the peptide solution, EDC (between 5-25 mM concentration), NHS (between 5-25 mM concentration) was dissolved in PBS and DMF (3:2, respectively). The pH was adjusted to 5 and the peptide was added to the solution (between 10 mg to 100 mg), and was allowed to mix for 3 hours. To activate the HPA solution, EDC (between 5-25 mM concentration), NHS (between 5-25 mM concentration) was dissolved in PBS and DMF (3:2, respectively). The pH was adjusted to 5 and HPA was added to the solution (between 10 mg to 4 g), and the mixture was allowed to mix for 3 hours. After 3 hours of mixing, the peptide solution was added to the dissolved gelatin. The pH is adjusted to 5 and allowed to react for another 3 hours with the gelatin. After the 3 hours of Gelatin/peptide mixing and reaction, the HPA solution was added to the solution and allowed to react overnight. The next day the solution was filtered and dialyzed using a tangential flow filtration system (2 kDa pore size). The resulting gel may be crosslinked by known methods using HRP and H₂O₂.

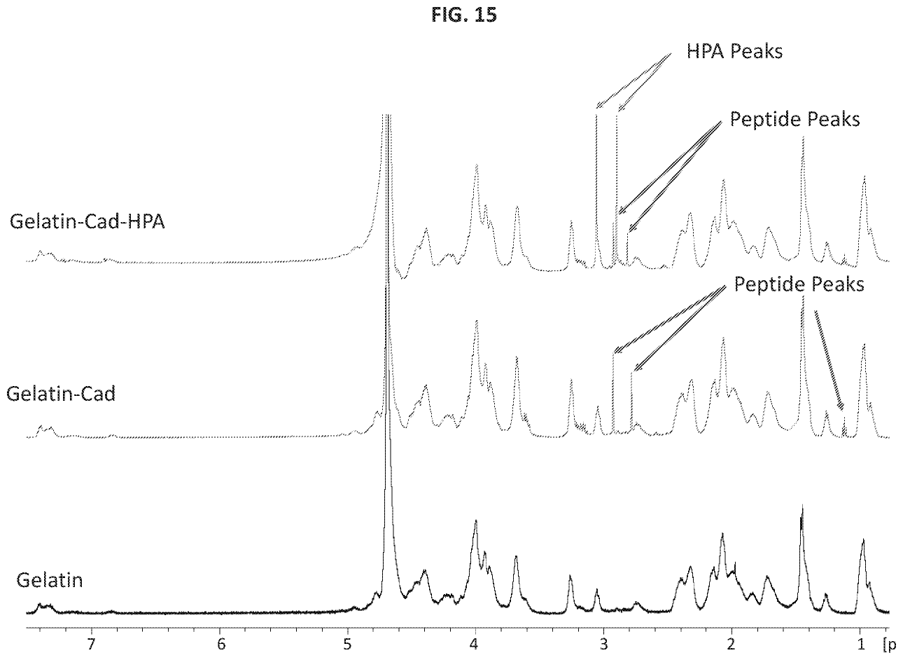

¹H-NMR spectrum showed the successful preparation of the resulting redox gel (Gelatin-Cad-HPA). The presence of HPA and Cad structures are confirmed by ¹H-NMR signals (˜1.10 and 2.50-3.10 ppm) (FIG. 15).

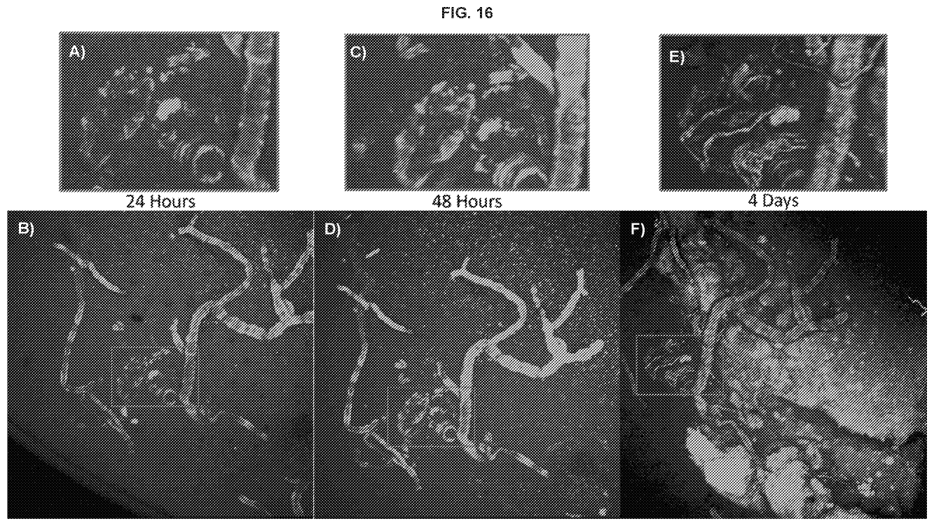

To determine whether redox-crosslinking hydrogels support vascular outgrowth in primary human brain tissue, cortex was dissected and embedded in a redox-crosslinking hydrogel. Fluorescent images show that new vessels sprout in primary human brain tissue when embedded in redox-crosslinking hydrogels (FIG. 16). Vascular outgrowth begins 24 hours after embedding in the hydrogel (FIG. 16A-16B). Vascular outgrowth continues throughout 48 hours (FIG. 16C-16D) and 4 days (FIG. 16E-16F) after embedding the brain tissue in the hydrogel. These data show that redox-crosslinking hydrogels support vascular sprouting in primary human brain tissue.

It is understood that the foregoing detailed description and accompanying examples are merely illustrative and are not to be taken as limitations upon the scope of the invention, which is defined solely by the following claims.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A biomaterial comprising a crosslinked hydrogel and a peptide chemically attached to the hydrogel, wherein the peptide comprises a histidine-alanine-valine (HAV) sequence.

Clause 2. The biomaterial of clause 1, wherein the peptide is attached to the hydrogel at the C-terminus.

Clause 3. The biomaterial of any one of clauses 1-2, wherein the peptide is 5 to 30 amino acids in length.

Clause 4. The biomaterial of any one of clauses 1-3, wherein the peptide comprises an extracellular epitope of a cadherin protein.

Clause 5. The biomaterial of any one of clauses 1-4, wherein the peptide further comprises a Asp-Ile-Gly-Gly (DIGG) sequence, a Asp-Ile-Asn-Gly (DING) sequence, a Ser-Ser-Asn-Gly (SSNG) sequence, or a Ser-Glu-Asn-Gly (SENG) sequence, wherein the DIGG, DING, SSNG, or SENG sequence is C-terminal to the HAV sequence.

Clause 6. The biomaterial of any one of clauses 1-5, wherein the peptide comprises SEQ ID NO: 1, SEQ ID NO: 2, or a variant thereof.

Clause 7. The biomaterial of any one of clauses 1-6, wherein the hydrogel is crosslinked by enzymatic crosslinking, thermal crosslinking, a crosslinker, or a combination thereof.

Clause 8. The biomaterial of any one of clauses 1-7, wherein the hydrogel is crosslinked by a crosslinker.

Clause 9. The biomaterial of any one of clauses 1-8, wherein the hydrogel is crosslinked by a UV-light activated crosslinker, a redox-activated crosslinker, or a combination thereof.

Clause 10. The biomaterial of any one of clauses 7-9, wherein the crosslinker comprises an optionally substituted vinyl group, an optionally substituted phenol group, or a combination thereof.

Clause 11. The biomaterial of clause 7-10, wherein the crosslinker comprises a —C(CH₃)═CH₂ group.

Clause 12. The biomaterial of clause 7-10, wherein the crosslinker comprises a phenol group.

Clause 13. The biomaterial of any one of clauses 1-12, wherein the hydrogel comprises gelatin.

Clause 14. The biomaterial of clause 13, wherein the gelatin comprises porcine skin gelatin.

Clause 15. The biomaterial of any one of clauses 1-14, wherein the biomaterial has a tunable stiffness about 800 Pa to about 5 kPa.

Clause 16. The biomaterial of any one of clauses 1-15, wherein the biomaterial has a pore sizes of about 20 μm to about 80 μm in diameter.

Clause 17. A method of preparing a biomaterial, comprising:

chemically attaching a peptide comprising a histidine-alanine-valine (HAV) sequence to a hydrogel; and

crosslinking the hydrogel having the attached peptide.

Clause 18. The method of clause 17, wherein the peptide comprises SEQ ID NO: 1, SEQ ID NO: 2, or a variant thereof.

Clause 19. The method of any one of clauses 17-18, wherein the crosslinking comprises enzymatic crosslinking, thermal crosslinking, chemically attaching a crosslinker to the hydrogel, or a combination thereof.

Clause 20. The method of any one of clauses 17-19, wherein the crosslinking comprises

chemically attaching a crosslinker to the hydrogel; and

crosslinking the hydrogel having the attached peptide and the attached crosslinker.

Clause 21. The method of any one of clauses 17-20, wherein the crosslinker comprise a UV-light activated crosslinker, a redox-activated crosslinker, or a combination thereof.

Clause 22. The method of any one of clauses 17-21, wherein the crosslinker comprises an optionally substituted vinyl group, an optionally substituted phenol group, or a combination thereof.

Clause 23. The method of clause 22, wherein the crosslinker comprises a —C(CH₃)═CH₂ group. For example, the crosslinker may be methacrylic acid.

Clause 24. The method of clause 22, wherein the crosslinker comprises a phenol group. For example, the crosslinker may be 3-(4-hydroxyphenyl)propionic acid.

Clause 25. The method of any one of clauses 17-24, wherein the hydrogel comprises gelatin.

Clause 26. The method of clause 25, wherein the gelatin comprises porcine skin gelatin.

Clause 27. A method of preparing a biomaterial, comprising:

chemically attaching methacrylic acid to a hydrogel to form a methacrylated hydrogel;

chemically attaching a peptide comprising a histidine-alanine-valine (HAV) sequence to the methacrylated hydrogel; and

exposing the resulting hydrogel to UV light, thereby causing the hydrogel to crosslink.

Clause 28. A method of preparing a biomaterial, comprising:

chemically attaching a peptide comprising a histidine-alanine-valine (HAV) sequence to form a functionalized hydrogel;

chemically attaching 3-(4-hydroxyphenyl)propionic acid to the functionalized hydrogel; and

subjecting the resulting hydrogel to an oxidation reaction, thereby causing the hydrogel to crosslink.

Clause 29. A biomaterial prepared by the method of any one of clauses 17, 27, and 28.

Clause 30. A method of culturing a plurality of cells, comprising contacting the plurality of cells with the biomaterial of clause 1 or clause 29.

Clause 31. The method of clause 30, wherein the cells are derived from induced pluripotent stem cells (iPSCs).

Clause 32. The method of any one of clauses 30-31, wherein the plurality of cells comprise a neuron, a brain endothelial cell, a glial cell, or a combination thereof.

Clause 33. The method of any one of clauses 30-32, wherein the plurality of cells comprise a neuron.

Clause 34. The method of any one of clauses 30-32, wherein the plurality of cells comprise a brain endothelial cell.

Clause 35. The method of any one of clauses 30-32, wherein the plurality of cells comprise a glial cell.

Clause 36. The method of any one of clauses 30-35, wherein the plurality of cells are differentiated into a brain organoid.

Clause 37. The biomaterial of clause 1 or clause 29, wherein a brain organoid is embedded in the biomaterial, wherein the biomaterial enables the brain organoid to be uniform and spherical.

Clause 38. The biomaterial of clause 37, wherein the brain organoid has laminar patterning of cortical layers.

Clause 39. The biomaterial of clause 1 or clause 29, wherein a tissue is embedded in the biomaterial.

Clause 40. The biomaterial of clause 39, wherein the biomaterial increases new blood vessel growth in the tissue.

Clause 41. The biomaterial of any one of clauses 39-40, wherein the tissue is mammalian tissue, fish tissue, reptilian tissue, bird tissue, amphibian tissue, or arthropod tissue.

Clause 42. The biomaterial of clause 41, wherein the tissue is human tissue.

Clause 43. The biomaterial of any one of clauses 39-42, wherein the tissue is brain tissue.

Clause 44. The biomaterial of any one of clauses 39-43, wherein the blood vessel is an artery, a capillary, an arteriole, a venule, a vein, or a combination thereof.

Clause 45. The biomaterial of any one of clauses 39-44, wherein the blood vessel comprises endothelial cells, wherein the endothelial cells maintain expression of vascular endothelial-cadherin.

### SEQUENCES

### SEQ ID NO: 1

### HAVDIGGGC

### SEQ ID NO: 2

### HAVDIGGGCE

### SEQ ID NO: 3

### AGVGDHIGC

What is claimed is:

1. A biomaterial comprising a crosslinked hydrogel and a peptide chemically attached to the hydrogel, wherein the peptide comprises a histidine-alanine-valine (HAV) sequence.

2. The biomaterial of claim 1, wherein the peptide is attached to the hydrogel at the C-terminal end.

3. The biomaterial of any one of claims 1-2, wherein the peptide is 5 to 30 amino acids in length.

4. The biomaterial of any one of claims 1-3, wherein the peptide comprises an extracellular epitope of a cadherin protein.

5. The biomaterial of any one of claims 1-4, wherein the peptide further comprises a Asp-Ile-Gly-Gly (DIGG) sequence, a Asp-Ile-Asn-Gly (DING) sequence, a Ser-Ser-Asn-Gly (SSNG) sequence, or a Ser-Glu-Asn-Gly (SENG) sequence, wherein the DIGG, DING, SSNG, or SENG sequence is C-terminal to the HAV sequence.

6. The biomaterial of any one of claims 1-5, wherein the peptide comprises SEQ ID NO: 1, SEQ ID NO: 2, or a variant thereof.

7. The biomaterial of any one of claims 1-6, wherein the hydrogel is crosslinked by enzymatic crosslinking, thermal crosslinking, a crosslinker, or a combination thereof.

8. The biomaterial of any one of claims 1-7, wherein the hydrogel is crosslinked by a crosslinker.

9. The biomaterial of any one of claims 1-8, wherein the hydrogel is crosslinked by a UV-light activated crosslinker, a redox-activated crosslinker, or a combination thereof.

10. The biomaterial of any one of claims 7-9, wherein the crosslinker comprises an optionally substituted vinyl group, an optionally substituted phenol group, or a combination thereof.

11. The biomaterial of claim 7-10, wherein the crosslinker comprises a —C(CH₃)═CH₂ group.

12. The biomaterial of claim 7-10, wherein the crosslinker comprises a phenol group.

13. The biomaterial of any one of claims 1-12, wherein the hydrogel comprises gelatin.

14. The biomaterial of claim 13, wherein the gelatin comprises porcine skin gelatin.

15. The biomaterial of any one of claims 1-14, wherein the biomaterial has a stiffness about 800 Pa to about 5 kPa.

16. The biomaterial of any one of claims 1-15, wherein the biomaterial has a pore size of about 20 μm to about 80 μm in diameter.

17. A method of preparing a biomaterial, comprising:
chemically attaching a peptide comprising a histidine-alanine-valine (HAV) sequence to a hydrogel; and
crosslinking the hydrogel having the attached peptide.

18. The method of claim 17, wherein the peptide comprises SEQ ID NO: 1, SEQ ID NO: 2, or a variant thereof.

19. The method of any one of claims 17-18, wherein the crosslinking comprises enzymatic crosslinking, thermal crosslinking, chemically attaching a crosslinker to the hydrogel, or a combination thereof.

20. The method of any one of claims 17-19, wherein the crosslinking comprises chemically attaching a crosslinker to the hydrogel; and
crosslinking the hydrogel having the attached peptide and the attached crosslinker.

21. The method of any one of claims 17-20, wherein the crosslinker comprise a UV-light activated crosslinker, a redox-activated crosslinker, or a combination thereof.

22. The method of any one of claims 17-21, wherein the crosslinker comprises an optionally substituted vinyl group, an optionally substituted phenol group, or a combination thereof.

23. The method of claim 22, wherein the crosslinker comprises a —C(CH₃)═CH₂ group.

24. The method of claim 22, wherein the crosslinker comprises a phenol group.

25. The method of any one of claims 17-24, wherein the hydrogel comprises gelatin.

26. The method of claim 25, wherein the gelatin comprises porcine skin gelatin.

27. A method of preparing a biomaterial, comprising:
chemically attaching methacrylic acid to a hydrogel to form a methacrylated hydrogel;
chemically attaching a peptide comprising a histidine-alanine-valine (HAV) sequence to the methacrylated hydrogel; and
exposing the resulting hydrogel to UV light, thereby causing the hydrogel to crosslink.

28. A method of preparing a biomaterial, comprising:
chemically attaching a peptide comprising a histidine-alanine-valine (HAV) sequence to form a functionalized hydrogel;
chemically attaching 3-(4-hydroxyphenyl)propionic acid to the functionalized hydrogel; and
subjecting the resulting hydrogel to an oxidation reaction, thereby causing the hydrogel to crosslink.

29. A biomaterial prepared by the method of any one of claims 17, 27, and 28.

30. A method of culturing a plurality of cells, comprising contacting the plurality of cells with the biomaterial of claim 1 or claim 29.

31. The method of claim 30, wherein the cells are derived from induced pluripotent stem cells (iPSCs).

32. The method of any one of claims 30-31, wherein the plurality of cells comprise a neuron, a brain endothelial cell, a glial cell, or a combination thereof.

33. The method of any one of claims 30-32, wherein the plurality of cells comprise a neuron.

34. The method of any one of claims 30-32, wherein the plurality of cells comprise a brain endothelial cell.

35. The method of any one of claims 30-32, wherein the plurality of cells comprise a glial cell.

36. The method of any one of claims 30-35, wherein the plurality of cells are differentiated into a brain organoid.

37. The biomaterial of claim 1 or claim 29, wherein a brain organoid is embedded in the biomaterial, wherein the biomaterial enables the brain organoid to be uniform and spherical.

38. The biomaterial of claim 37, wherein the brain organoid has laminar patterning of cortical layers.

39. The biomaterial of claim 1 or claim 29, wherein a tissue is embedded in the biomaterial.

40. The biomaterial of claim 39, wherein the biomaterial increases new blood vessel growth in the tissue.

41. The biomaterial of any one of claims 39-40, wherein the tissue is mammalian tissue, fish tissue, reptilian tissue, bird tissue, amphibian tissue, or arthropod tissue.

42. The biomaterial of claim 41, wherein the tissue is human tissue.

43. The biomaterial of any one of claims 39-42, wherein the tissue is brain tissue.

44. The biomaterial of any one of claims 39-43, wherein the blood vessel is an artery, a capillary, an arteriole, a venule, a vein, or a combination thereof.

45. The biomaterial of any one of claims 39-44, wherein the blood vessel comprises endothelial cells, wherein the endothelial cells maintain expression of vascular endothelial-cadherin.


SEQUENCE LISTING (3 sequences)

SEQ ID NO: 1 - protein, 9 aa, Artificial Sequence
  Synthetic
HAVDIGGGC

SEQ ID NO: 2 - protein, 10 aa, Artificial Sequence
  Synthetic
HAVDIGGGCE

SEQ ID NO: 3 - protein, 9 aa, Artificial Sequence
  Synthetic
AGVGDHIGC